(19) United States
(12) Patent Application Publication
(10) Pub. No.: US 2003/0054332 A1
(43) Pub. Date: Mar. 20, 2003

(54) Markers for evaluating estrogenic activity

(76) Inventors: Miguel S. Barbosa, Claremont, CA (US); Helen A. Brady, San Diego, CA (US); Kyle W.H. Chan, San Diego, CA (US); Jose R. Pardinas, Bothell, WA (US)

Correspondence Address: PENNIE AND EDMONDS, 1155 AVENUE OF THE AMERICAS, NEW YORK, NY, 100362711

(21) Appl. No.: 09/853,544

(22) Filed: May 10, 2001

(51) Int. Cl. C12Q 1/00; C12Q 1/68; C07H 21/04; C12N 5/06

(52) U.S. Cl. 435/4; 435/6; 435/325; 536/23.5

(57) ABSTRACT

Methods are provided for evaluating estrogenic and antiestrogenic effects of candidate therapeutic agents. Such methods are generally based on assays to detect modulation of estrogen-regulated marker expression in one or more specific cell types. Agents identified using such methods may be used, for example, in the prevention and treatmnt of diseases such as osteoporosis, cardiovascular disease and cancer.

In addition, the gene discovery approaches discussed have identified a unique gene profile for estrogen regulation in vascular endothelial cells. This gene profile will allow characterization of the effects of potential SERMs in the cardiovascular system. This gene profile and the assays established with these genes will enable more extensive evaluation of tissue specific properties of SERM compounds and provide a better understanding of cardiovascular effects of SERMs.

### TECHNICAL FIELD

The present invention relates generally to the identification of compounds that have estrogenic and/or antiestrogenic activity, such as cell-type specific estrogenic agents. The invention is more particularly related to screens for use in evaluating compounds for cardioprotective and bone protective estrogenic effects, and to methods for using such compounds to prevent or treat diseases such as cardiovascular disease, osteoporosis and cancer.

### BACKGROUND OF THE INVENTION

Estrogen is a therapeutic component of hormone replacement therapy (HRT), which is commonly used to treat symptoms associated with the onset of menopause. This hormone has further been shown to provide protection from cardiovascular disease, which is a leading cause of death among American women. While the mechanism of this effect is not understood, estrogens appear to decrease the accumulation of low-density lipoprotein and cholesterol in coronary arteries, and to increase the level of high-density lipoprotein. Estrogens also appear to function as vasodilators and antioxidants, which may further provide some cardioprotective effect. Other positive results of estrogen therapy include protection from osteoporosis, as well as beneficial neuronal effects. However, in spite of the benefits of estrogen treatment, many women are concerned about the threat of increased incidence of breast and/or uterine cancer.

A drug that provides the many benefits of estrogen with minimal or decreased risk of cancer would alleviate such fears and improve the health of many women. In attempts to identify such a drug, compounds called Selective Estrogen Receptor Modulators (SERMs) have been pursued. SERMs are compounds that can have estrogenic activity in one tissue, while having no estrogenic activity or acting as an estrogen antagonist in another tissue. For example, such an agent could have estrogenic activity in the bone and cardiovascular system (thus preventing osteoporosis and cardiovascular disease), but act as an antiestrogen in breast and uterus (thus reducing the risk of breast and endometrial cancer). The use of SERMs clearly offers promise as breast cancer preventative agents, and such compounds have the potential to reduce the occurrences of uterine cancer, osteoporosis and myocardial infarction.

A few candidate SERMs are in clinical use or in some stage of clinical development. Tamoxifen, for example, has been used for a number of years in cancer treatment and recently won FDA approval for use in breast cancer prevention in high-risk women. Raloxifene has been approved for use in prevention of osteoporosis. Both compounds were first identified as “antiestrogens” due to their estrogen antagonistic activity in the breast. However, studies of the role of these compounds in cancer prevention have lead to conflicting conclusions. Of particular concern is the effect of these compounds on uterine proliferation and endometrial cancer. In addition, the cardioprotective effects of both compounds need further evaluation.

A significant limitation in the identification and evaluation of candidate SERMs is the lack of convenient screens. Currently, time-consuming and labor-intensive clinical trials are needed to assess the activities of an individual candidate. In order to identify a compound that has estrogen's positive effects on bone and the cardiovascular system, while protecting against estrogen's negative effects on breast and endometrial cancer, techniques are needed to provide information about a potential SERM's tissue specific molecular profile prior to in vivo and clinical studies.

Accordingly, there is a need in the art for a system that permits a rapid assay for cell type-specific effects of estrogenic therapeutic agents. The present invention fulfills this need and further provides other related advantages.

### SUMMARY OF THE INVENTION

Briefly stated, the present invention provides screens for use in evaluating compounds for cardiovascular effects, and methods for preventing and treating cardiovascular disease. Within certain aspects, the present invention provides methods for evaluating the cardiovascular effect of a candidate agent, comprising the steps of: (a) contacting an estrogen receptor-enhanced vascular endothelial cell with a candidate agent; and (b) determining whether the candidate agent modulates expression of at least one estrogen-regulated marker, wherein the expression of the marker is previously determined to be induced by estrogen in estrogen receptor-enhanced vascular endothelial cells. In certain embodiments, step (b) may comprise the steps of: (i) detecting a level of an estrogen-regulated marker, and (ii) comparing the detected level of the estrogen-regulated marker with a level of the estrogen-regulated marker in the absence of the candidate agent; or the steps of (i) detecting a level of mRNA encoding an estrogen-regulated marker, and (ii) comparing the detected level of mRNA encoding the estrogen-regulated marker with a level of mRNA encoding the estrogen-regulated marker in the absence of estrogen-related compound. In certain other embodiments, the cell is transfected with a polynucleotide comprising a promoter for an estrogen-regulated marker, operably linked to a reporter gene. Within such embodiments, step (b) may comprise the steps of: (i) detecting a level of reporter protein activity, and (ii) comparing the level of reporter protein activity detected with a level of reporter protein in the absence of candidate agent, or may comprise the steps of: (i) detecting a level of reporter mRNA, and (ii) comparing the level of reporter mRNA detected with a level of reporter mRNA in the absence of estrogen-related compound. Certain methods evaluate the ability of the candidate agent to modulate expression of at least two estrogen-regulated markers (ERMs). Preferred markers are those listed in Table I, i.e., ERM-1, ERM-2, ERM-3, ERM-4, ERM-5, ERM-6, ERM-7, ERM-8, ERM-9, ERM-10, ERM-11, ERM-12, ERM-13, ERM-14, ERM-15, ERM-16, ERM-17, ERM-18, ERM-19, ERM-20, ERM-21, ERM-22, ERM-23, ERM-24, ERM-25, ERM-26, ERM-27, ERM-28, ERM-29, ERM-30, ERM-31, ERM-32, ERM-33, ERM-34, ERM-35, ERM-36, ERM-37, ERM-38, ERM-39, ERM-40, ERM-41, ERM-42, ERM-43, ERM-44, ERM-45, ERM-46, ERM-47, ERM-48, ERM-49, ERM-50, ERM-51, ERM-52, ERM-53, ERM-54, ERM-55, ERM-56, ERM-57, ERM-58, ERM-60, ERM-61, ERM-62, ERM-63, ERM-64, ERM-65, ERM-66, ERM-67, ERM-68, ERM-69, ERM-70, ERM-71, ERM-72, ERM-73, ERM-74 and ERM-75. Preferably, the ERM is coded for by a message that hybridizes under stringent conditions to any one if SEQ ID NOs:1-75. Preferred estrogen-regulated markers are those having sequences recited in any one of SEQ ID NOs:1-75.

Within further aspects, methods are provided for identifying a selective estrogen receptor modulator (SERM), comprising the steps of: (a) contacting at least two different cells that express an estrogen-regulated marker with a candidate SERM; (b) determining levels of expression of at least one estrogen-regulated marker in the cells; and (c) comparing the levels of expression with levels of expression of the estrogen-regulated marker(s) in cells following contact with estrogen, and therefrom determining whether the candidate SERM is a selective estrogen receptor modulator. The estrogen-regulated marker may be, for example, partially or fully encoded by one of the nucleic acids sequences set forth in SEQ ID NOs:1-75, or by a fragment of one of the nucleic acid sequences set forth in SEQ ID NOs: 1-75, or by a nucleic acid that hybridizes under stringent conditions to one of the nucleic acid sequences set forth in SEQ ID NOs: 1-75, and the cells may be independently selected from the group consisting of breast, endometrial, cardiovascular, bone and central nervous system cells. Within certain embodiments, levels of at least two estrogen-regulated markers are determined. Certain methods further comprise the steps of: (d) contacting breast cells that express an estrogen-regulated marker expression with estrogen and a candidate SERM; (e) determining levels of expression of at least one estrogen-regulated marker in the breast cells; and (f) comparing the levels of expression with levels of expression of the estrogen-regulated marker(s) in the breast cells following contact with estrogen, in the absence of candidate SERM.

Within further aspects, methods are provided for identifying estrogen regulated markers, comprising the steps of: (a) contacting a cell that expresses an estrogen receptor with an estrogenic compound for a period of time (b) isolating RNA from said cells; (c) determining levels of expression of one or more RNA molecules; and (d) comparing the levels of expression with levels of expression of the RNA molecules in cells not contacted with estrogen, and therefrom determining whether the one or more RNA molecules are estrogen regulated markers. The level of expression of RNA molecules can be determined by, for example, RNA fingerprinting or by probing arrays, such as those provided by the Incyte Genomics UniGEM technology.

Within further aspects, methods are provided for inhibiting the development of a cardiovascular disease in a patient; comprising administering to a patient an agent identified or a SERM identified as described above.

Methods are also provided, within other aspects, for inhibiting the development of breast cancer in a patient; comprising administering to a patient a SERM identified as described above, wherein the SERM inhibits induction of estrogen-regulated marker expression by estrogen in breast cells.

The present invention further provides, within other aspects, methods for inhibiting the development of osteoporosis in a patient; comprising administering to a patient a SERM identified as described above.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS

FIG. 1 is an autoradiogram depicting the level of expression of vascular endothelial growth factor (VEGF) mRNA in VE-ERα cells following exposure to 100 nM 17,β-estradiol for varying amounts of time, as indicated. Vehicle (ethanol) controls are shown in the “−” lanes. GADPH expression is also shown for reference.

FIG. 2 is an autoradiogram depicting the level of prostacyclin I 2 synthase in VE-ERα cells following exposure to 100 nM 17,β-estradiol for varying amounts of time, as indicated, in the lanes labeled “E2”. Vehicle (ethanol) controls are shown in the “EtOH” lanes. GADPH expression is also shown for reference.

FIG. 3 is an autoradiogram depicting the time course of cyclin A1 induction in estrogen receptor α-enhanced vascular endothelial cells, as determined by RNase protection analyses. Cells were treated by adding 0.1% ethanol (ctrl) or 100 nM 17-β-estradiol (E2) for the indicated times. mRNA levels of cyclin A1 and glyceraldehyde-3-phosphate dehydrogenase (GADPH) are shown as indicated.

FIGS. 4A and 4B are autoradiograms illustrating RNase protection experiments with estrogen receptor α- and estrogen receptor β-enhanced vascular endothelial cells. Cells were treated by adding 0.1% ethanol (ctrl), 100 nM 17-β-estradiol (E2), 1 μM ICI 164384 (ICI) or 10 nM diethylstilbestrol (DES), as indicated. In each case, the levels of various cyclins are shown, as indicated by the designations to the right of the autoradiogram.

FIG. 5 is an autoradiogram depicting the effect of varying amounts of 17-β-estradiol on the levels of various estrogen-regulated markers (as indicated). The GAPDH control is labeled and DDST is diastrophic dysplasia sulfate transporter.

### DETAILED DESCRIPTION OF THE INVENTION

As noted above, the present invention is generally directed to screens for use in evaluating compounds for estrogenic effects, such as cell-type specific and/or cardioprotective effects. Compounds that may be identified using such methods include those with mixed agonist and antagonist properties, which can be used in prevention of breast cancer while also having estrogen-like positive effects in bone and the cardiovascular system.

The present invention is based, in part, on the identification of estrogen-regulated markers, which can be used to evaluate candidate agents for cell type-specific estrogenic activity and cardioprotective effects. Such markers may be used, for example, to obtain estrogen-regulated marker expression profiles in tissues of interest (e.g., breast, endometrium, bone, central nervous system and cardiovascular tissue). Such profiles may be used to evaluate the estrogenic activity of a candidate agent in various cell types, thereby permitting rapid screening of candidate agents for selective estrogen receptor modulator (SERM) activity.

Estrogen-Regulated Markers (ERMs)

An “estrogen-regulated marker,” or “ERM,” as used herein is a gene which has an altered level of expression, either increased or decreased, preferably at least two fold greater, in response to estrogen in estrogen receptor-expressing cells, such as estrogen receptor-enhanced vascular endothelial cells or mammary epithelial cells. As used herein, the term “estrogen receptor-enhanced” refers to cells that express an estrogen receptor (e.g., estrogen receptor alpha (ERα) and/or estrogen receptor beta (ERβ)) at a level that is sufficient to permit the cells to respond to estrogen using any assay that measures an estrogen response. Vascular endothelial and mammary epithelial cells, in culture, do not naturally express estrogen receptors at such a level. However, such cells may be rendered estrogen receptor-enhanced by transfection with one or more estrogen receptor genes, as discussed herein. Assays to detect an estrogen response include assays to detect enhanced levels of any estrogen responsive gene i.e. vascular endothelial growth factor (VEGF) mRNA following treatment with estrogen, as well as assays to detect the increases in the expression of an estrogen-regulated marker or ERM related protein as provided herein.

The term “ERM related protein” as used herein refers to a polypeptide that possesses a similar or identical amino acid sequence to that of an ERM, or possesses a structure that is similar or identical to that of the ERM. As used herein, an amino acid sequence of a polypeptide is “similar” to that of an ERM if it satisfies at least one of the following criteria: (a) the polypeptide has an amino acid sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the amino acid sequence of the ERM; (b) the polypeptide is encoded by a nucleotide sequence that hybridizes under stringent conditions to a nucleotide sequence encoding at least 5 amino acid residues (more preferably, at least 10 amino acid residues, at least 15 amino acid residues, at least 20 amino acid residues, at least 25 amino acid residues, at least 40 amino acid residues, at least 50 amino acid residues, at least 60 amino residues, at least 70 amino acid residues, at least 80 amino acid residues, at least 90 amino acid residues, at least 100 amino acid residues, at least 125 amino acid residues, or at least 150 amino acid residues) of the ERM; or (c) the polypeptide is encoded by a nucleotide sequence that is at least 30% (more preferably, at least 35%, at least 40%, at least 45%, at least 50%, at least 55%, at least 60%, at least 65%, at least 70%, at least 75%, at least 80%, at least 85%, at least 90%, at least 95% or at least 99%) identical to the nucleotide sequence encoding the ERM.

For a high degree of selectivity, relatively stringent conditions are used to form the duplexes, such as low salt or high temperature conditions. As used herein, “highly stringent conditions” means hybridization to filter-bound DNA in 0.5 M NaHPO₄, 7% sodium dodecyl sulfate (SDS), 1 mM EDTA at 65° C., and washing in 0.1× SSC/0.1% SDS at 68° C. (Ausubel F. M. et al., eds., 1989, Current Protocols in Molecular Biology, Vol. I, Green Publishing Associates, Inc., and John Wiley & Sons, Inc., New York, at p. 2.10.3; incorporated herein by reference in its entirety.) For some applications, less stringent conditions for duplex formation are required. As used herein “moderately stringent conditions” means washing in 0.2× SSC/0.1% SDS at 42° C. (Ausubel et al., 1989, supra). Hybridization conditions can also be rendered more stringent by the addition of increasing amounts of formamide, to destabilize the hybrid duplex. Thus, particular hybridization conditions can be readily manipulated, and will generally be chosen depending on the desired results. In general, convenient hybridization temperatures in the presence of 50% formamide are: 42° C. for a probe which is 95 to 100% identical to the fragment of a gene encoding an ERM, 37° C. for 90 to 95% identity and 32° C. for 70 to 90% identity.

In general, estrogen-regulated marker expression is detectably changed in an estrogen receptor-enhanced vascular endothelial cell or mammary epithelial cell treated with estrogen as described herein, whereas expression of the marker does not respond detectably to estrogen in such cells without estrogen-receptor enhancement. Estrogen receptor-enhanced cells may be derived from primary cells obtained from a human or non-human animal, or may be derived from a cell line. For example, primary vascular endothelial cells or mammary epithelial cells may be infected with an immortalizing retrovirus (i.e., a retrovirus that expresses an oncogene that leads to immortalization of infected cells). Such immortalized cells have very low expression of estrogen receptors and no detectable estrogen response. Estrogen receptor enhancement may then be achieved by infection with a retrovirus that expresses recombinant estrogen receptor a or estrogen receptor β. Infected cells may generally be selected based on antibiotic resistance. The level of estrogen receptor mRNA may be confirmed using, for example, an RNase protection assay, and the level of estrogen receptor protein determined by Western analysis or immunohistochemical techniques.

Estrogen-regulated markers may be identified in such cells using any technique that is suitable for detecting differential gene expression. There are numerous such techniques that are well known in the art. One convenient screening method involves the use of commercially available chips containing large numbers of human sequences (e.g., the UniGEM technology, available from Incyte). Briefly, polyA RNA is isolated from cells treated with estrogen and from cells treated with a vehicle control. The RNA is used to generate labeled cDNA probes, which are then hybridized to the immobilized human sequences. In a preferred embodiment, sequences that are expressed at a level that is at least two fold greater in the presence of estrogen than in the absence of estrogen are considered estrogen-regulated markers, although sequences with any detectable increase or decrease may be used as markers. The estrogen-regulated marker may be, for example, partially or fully encoded by one of the nucleic acids sequences set forth in SEQ ID NOs:1-75, or by a fragment of one of the nucleic acid sequences set forth in SEQ ID NOs: 1-75, or by a nucleic acid that hybridizes under stringent conditions to one of the nucleic acid sequences set forth in SEQ ID Nos: 1-75. Within the context of the present invention, the sequences listed in Table I (see Example 2) have been found to encode estrogen-regulated markers. Preferred estrogen-regulated markers include, for example, cyclin A1, prostacyclin (PGI2) synthase, connexin 37, diastrophic dysplasia sulfate transporter, podocalyxin, and clusterin. Prefered estrogen-regulated markers are selected based on biological relevence, fold activation and other factors. It will be apparent, however, that other estrogen-regulated markers may be identified using the methods provided herein, and may be used as markers within the screens discussed below.

Assays for the Identification of Therapeutic Agents

The estrogen-regulated markers provided herein may be used within assays designed to identify therapeutic agents with estrogenic and/or antiestrogenic (i.e., estrogen antagonistic) activity. Candidate compounds for use within the assays provided herein may be derived from any source, including small molecule combinatorial libraries. Certain candidate compounds are estrogen-related. As used herein, an “estrogen-related compound” is any compound that binds detectably to an estrogen receptor. Such binding may be detected using any assay known in the art, including direct binding assays and functional assays (e.g., assays for the ability to stimulate VEGF expression).

Within certain assays, estrogen-regulated markers, ERM related proteins, or nucleic acids encoding ERMs or ERM related proteins may be used to identify selective estrogen receptor modulators (referred to herein as SERMs). Known SERMs include Raloxifene (Evista); Tamoxifen(Nolvadex); Toremifene(Fareston); Idoxifene; Droloxifene; Tibolone(Livial); Levormeloxifene; GW-5638; CP-336,156 (Lasofoxifene); ICI-182,780 (Faslodex); EM-800 (EM-652); TAT-59 (Miproxifene); Compound 7b (cyclopropyl antiestrogen); Analog II (diarylcyclopropane); ERA-923; TSE-424; SR 16234, SR16287; LY353381; HMR-3339; EM-800. In preferred embodiments, the methods of the invention do not include the administration, identification or screening for any of these recited, known SERMs. In preferred embodiments, the compositions of the invention do not include the presence of a recited, known SERM.

Within such assays, the effect of a candidate modulator on an expression profile of one or more estrogen-regulated markers or ERM related proteins or nucleic acid encoding therefor is determined. In a preferred embodiment, the detected marker is or comprises a fragment of one or more of the following: ERM-1, ERM-2, ERM-3, ERM-4, ERM-5, ERM-6, ERM-7, ERM-8, ERM-9, ERM-10, ERM-11, ERM-12, ERM-13, ERM-14, ERM-15, ERM-16, ERM-17, ERM-18, ERM-19, ERM-20, ERM-21, ERM-22, ERM-23, ERM-24, ERM-25, ERM-26, ERM-27, ERM-28, ERM-29, ERM-30, ERM-31, ERM-32, ERM-33, ERM-34, ERM-35, ERM-36, ERM-37, ERM-38, ERM-39, ERM-40, ERM-41, ERM-42, ERM-43, ERM-44, ERM-45, ERM-46, ERM-47, ERM-48, ERM-49, ERM-50, ERM-51, ERM-52, ERM-53, ERM-54, ERM-55, ERM-56, ERM-57, ERM-58, ERM-60, ERM-61, ERM-62, ERM-63, ERM-64, ERM-65, ERM-66, ERM-67, ERM-68, ERM-69, ERM-70, ERM-71, ERM-72, ERM-73, ERM-74 and ERM-75. In another preferred embodiment, one or more estrogen-regulated markers are detected by hybridization under highly stringent conditions of one or more of SEQ ID Nos: 1-75 to mRNA from a cell of interest. An expression profile may be obtained using two or more cell types of interest. Briefly, the effect of estrogen on expression of an estrogen-regulated marker or ERM related protein, or nucleic acid coding therefor is determined in the selected cell types. The effect of a comparable amount of the candidate modulator is then determined and, for each cell type, the effect of the candidate modulator is compared to the effect of the estrogen.

More specifically, cell-types to be used within an expression profile are selected to permit the identification of SERMs that have estrogenic activity in tissues where such activity is beneficial (e.g., bone, central nervous system, urogenital tissue and cardiovascular tissue), and that have no activity or anti-estrogenic activity in tissues where estrogenic activity is potentially harmful (e.g., breast, ovarian and endometrial tissue). Preferred cell types for use in obtaining an expression profile include commercially available cells such as mammary gland adenocarcinoma cells, e.g., MCF-7 cells; mammary gland primary ductal carcinoma, e.g., HCC70, HCC1395, or HCC1500 cells, uterine sarcoma cells, e.g., MES-SA cells, cervical squamous cell carcinoma cells, e.g., SW756 cells, or human umbilical cord vascular enthothelial cells, e.g., HUVECs cells. Other cell lines which have been developed include endometrial epithelial, endometrial stromal, and osteoblast cells. Cells of nearly any type can be produced by, for example, obtaining early passage primary cells from commercial sources (e.g. Cell Applications, Inc., San Diego, Calif.) and infecting them with the immortalizing retrovirus LXSN16E6E7. Immortalized cells are selected by G418 treatment and determined to be growth extended relative to uninfected cells.

An expression profile of one or more ERMs, ERM related proteins or nucleic acids coding therefor may be obtained in a variety of ways, including measurement of the level of ERMs or ERM related proteins, or mRNA encoding the ERMs or ERM related proteins, in the selected cells. For example, antibodies specific for the protein of interest may be used to measure the level of expression of a protein of interest in, for example, an ELISA or Western blot. For several of the ERMs identified herein, antibodies are commercially available, and methods of making antibodies that recognize a protein are known to those of skill in the art. Polycolonal or monoclonal antibodies may be utilized.

Alternatively, the endogenous promoter for the gene encoding the ERM or ERM related protein may be isolated and operably linked to a reporter gene. Reporter genes include the luciferase gene, CAT, or any other reporter known to those of skill in the art. Cells transformed with such a construct may be used to generate an expression profile based on the level of reporter protein or mRNA in the cells.

Assays for qualitatively and quantitatively determining mRNA and protein levels in a sample are well known in the art and amply described in the patent and scientific literature. For example, expression may be determined at the transcriptional level (i.e., using any of a variety of well known hybridization or amplification techniques to assess the level of estrogen-regulated marker mRNA) or at the translational level (i.e., using a compound, such as an antibody, that binds to estrogen-regulated marker to assess the level of such protein synthesized in any standard assay format, such as ELISA). Representative methods are also provided herein.

After obtaining an expression profile, the effect of estrogen on the expression profile is generally determined. Any active form of estrogen may be used, such as 17-β estradiol or diethylstilbesterol (DES), generally in an amount ranging from 1 nM to 100 nM (preferably about 10 nM) for about 1-24 hours Following exposure of the cells to the estrogen, the expression profile for the estrogen-regulated marker is again determined, and the effect of estrogen on marker expression in each cell type is obtained. Optionally, the effect of estrogen on ERMs or ERM related proteins can be determined after exposure of the test cells to an effector molecule or protein other than an estrogenic one, such as TNF or IL-1. Thus, in one embodiment, cells are first contacted with a cytokine or other molecule with biological effect, such as TNF or IL-1, then contacted with an estrogenic molecule, such as DES. Expression profiles can be determined prior to contact with any effector molecule, after contact with the non-estrogenic effector molecule, and after contact with the estrogenic molecule.

Candidate SERMs may be evaluated in a similar manner. The cell types used in the estrogen-induced expression profile are contacted with the candidate SERM (generally in an amount ranging from 1 nM to 1000 nM, preferably 1 nM to 100 nM) for a period of time that is substantially similar to that used for exposure to estrogen (i.e., within about 10% of the estrogen contact time). Following exposure of the cells to the candidate SERM, the expression profile for the estrogen-regulated marker is determined, and the effect of the candidate SERM on marker expression in each cell type is obtained. Finally, for each cell type, the effect of the candidate SERM is compared to the effect of estrogen and other SERMS (i.e. tamoxifen, raloxifene and ICI 164,384) on marker expression. These other SERMs act by binding to the ligand binding site of the estrogen receptor. This is not a necessary criteria for a SERM. Any compound which alters activity of the estrogen receptor in the desired tissue(s) (i.e. increasing activity in tissues which estrogenic activity is desirable and decreasing where estrogenic activity is detrimental) would qualify. This would include compounds functioning by altering formation of the estrogen receptor complex (i.e. interactions between the estrogen receptor and coactivators and corepressors). A SERM is any candidate for which (a) the effect in test cells where estrogenic activity is desired is not substantially lower than the effect of estrogen (i.e., activity is greater, the same or at least 90%, 80%, 70%, 50%, 40%, or 30% of the activity of estrogen) and (b) the effect in test cells where estrogenic activity is undesirable is minimal (i.e., no more than 10%, 20%, or 30% of the activity of estrogen), undetectable or antiestrogenic.

For certain uses, a SERM preferably has antiestrogenic activity in certain cells (e.g., breast and endometrial). Antiestrogenic activity may be detected using the assays described above, with the modification that the level(s) of estrogen-regulated marker(s) are detected in the presence of both estrogen and candidate SERM, and compared to the levels detected in the presence of estrogen and the absence of candidate SERM. A SERM has antiestrogenic activity if contact with the SERM results in a decrease in estrogen-induced expression of the estrogen-regulated marker(s). Preferably, the decrease is at least 50% of the level of induction by estrogen.

Other assays may be used to evaluate the cardiovascular effect of a candidate agent. Briefly, the effect of a candidate agent on estrogen-regulated marker expression is determined in estrogen receptor-enhanced vascular endothelial cells. Estrogen-regulated marker expression may be evaluated in such cells by determining the level of marker mRNA or protein present, as discussed above. Alternatively, as discussed above, cells for use in such assays may be transfected with a plasmid comprising an estrogen-regulated marker promoter operably linked to a reporter gene. In such transfected cells, marker expression may be determined by evaluating reporter gene expression (i.e., measuring the level of reporter gene mRNA, reporter protein or reporter activity), as described above. Such screens may be used, for example, to identify estrogen-related compounds that have minimal cardiovascular effects, or to identify drugs for preventing and/or treating cardiovascular disease.

More specifically, to evaluate the cardiovascular effect of a candidate agent, estrogen-regulated marker expression is generally evaluated by any assay described herein within an estrogen receptor-enhanced vascular endothelial cell in the presence of the compound. Such expression is then compared to expression of the marker observed in the absence of candidate agent. Within certain preferred embodiments, estrogen-regulated marker expression is determined using real-time PCR analyses performed on RNA obtained from cells that were exposed to one or more candidate agents, as well as cells that were not exposed to such a compound. A compound that enhances estrogen-regulated marker expression may have a cardioprotective effect similar to that of estrogen. Such compounds may be of therapeutic value in treating certain cardiovascular diseases, or may be used prophylactically to inhibit the development of such diseases.

Another preferred assay is an RNase protection assay (RPA), which can be used to examine a panel of genes identified as estrogen-regulated in VE-ERα cells. The RPA is described in Gilman, 1993, In Current Protocols in Molecular Biology, Vol. 1 (Ausubel, F. M., R. Brent, R, E. Kingston, D. D. Moore, J. G. Seidman, J. A. Smith and K. Stuhl, eds.) pp. 4.7.1-4.7.8, John Wiley and Sons, Inc., New York, incorporated herein by reference in its entirety. Within this assay, which may be performed according to standard protocols, multiple probes can be used in a single reaction that requires as little as 1 μg of total RNA, resulting in high information content and a molecule profile for each compound on ERα. regulated gene expression in vascular endothelial cells. The RPA assay is very sensitive and is therefore able to detect low copy messages and distinguish low fold modulations typical of estrogen. This approach has a number of advantages over a reporter assay. The endogenous genes in the multigene assay will contain all endogenous gene regulatory elements and will not be restricted to activation occurring through an ERE element. Since VE-ERα cells are human cells that have been characterized and determined to maintain endothelial specific phenotypic markers and functions, tissue selective ER regulation can be investigated.

Within further aspects, methods are provided for identifying estrogen regulated markers, comprising the steps of: (a) contacting a cell that expresses an estrogen receptor with an estrogenic compound for a period of time (b) isolating RNA from said cells; (c) determining levels of expression of one or more RNA molecules; and (d) comparing the levels of expression with levels of expression of the RNA molecules in cells not contacted with estrogen, and therefrom determining whether the one or more RNA molecules are estrogen regulated markers. The level of expression of RNA molecules can be determined by, for example, RNA fingerprinting or by probing arrays, such as those provided by the Incyte Genomics UniGEM technology. The cell may be one that normally expresses an estrogen receptor, or one that has been genetically engineered to express an estrogen receptor.

ERM Peptides and Nucleic Acids

The invention features nucleic acid molecules which are at least 30%, 35%, 40%, 45%, 50%, 55%, 65%, 75%, 85%, 95%, or 98% identical to the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof. Preferably, the nucleic acid molecules are at least 30%, 35%, 40%, 45%, 50%, 55%, 65%, 75%, 85%, 95%, or 98% identical to at least or about 50, 100, 150, 200 or 250 consecutive nucleic acids of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof.

The invention features isolated polypeptides or proteins which are encoded by a nucleic acid molecule having or comprising a nucleotide sequence that is at least about 30%, preferably 35%, 40%, 45%, 50%, 55%, 65%, 75%, 85%, 95%, or 98% identical to the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, a complement thereof, or the non-coding strand thereof, wherein the polypeptides or proteins also exhibit at least one structural and/or functional feature of a polypeptide of the invention.

The invention features nucleic acid molecules of at least 50, 100, 150, 200, or 250 nucleotides of the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof.

The invention features isolated polypeptides or proteins which are encoded by a nucleic acid molecule having or comprising a nucleotide sequence that is at least about 50, 100, 150, 200, or 250 or more contiguous nucleotides identical to the nucleic acid sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof. Preferably, the polypeptides or proteins also exhibit at least one structural and/or functional feature of a polypeptide of the invention.

The invention also features nucleic acid molecules which include a nucleotide sequence encoding a protein having an amino acid sequence that is at least 25%, 30%, 35%, 40%, 45%, 50%, 55%, 60%, 65%, 75%, 85%, 95%, or 98% identical to the amino acid sequence of a protein coded for by any nucleic acid sequence recited or referred to by accession number in Table I.

In preferred embodiments, the nucleic acid molecules have the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I.

Also within the invention are nucleic acid molecules which encode a polypeptide having the amino acid sequence of a protein coded for by any nucleic acid sequence recited or referred to by accession number in Table I, or a fragment thereof including at least 10, 15, 20, 25, 30, 50, 75 or 80 contiguous amino acids thereof.

The invention also features nucleic acid molecules which encode a polypeptide fragment of at least 10, 15, 25, 30, 50, 75, 80 or more contiguous amino acids of a protein coded for by any nucleic acid sequence recited or referred to by accession number in Table I. Preferably, the fragment exhibits at least one structural and/or functional feature of a polypeptide of the invention.

The invention includes nucleic acid molecules which encode a naturally occurring allelic variant of a polypeptide comprising the amino acid sequence of a protein coded for by any nucleic acid sequence recited or referred to by accession number in Table I.

Also within the invention are isolated polypeptides or proteins having an amino acid sequence that is at least about 30%, preferably 40%, 45%, 55%, 65%, 75%, 85%, 95%, or 98% identical to the amino acid sequence of a protein coded for by any nucleic acid sequence recited or referred to by accession number in Table I.

Also within the invention are isolated polypeptides or proteins which are encoded by a nucleic acid molecule having a nucleotide sequence that is at least about 30%, preferably 35%, 40%, 45%, 50%, 55%, 60%, 65%, 75%, 85%, 95% or 98% identical to the nucleic acid sequence encoding a protein coded for by any nucleic acid sequence recited or referred to by accession number in Table I, and isolated polypeptides or proteins which are encoded by a nucleic acid molecule having a nucleotide sequence which hybridizes under stringent hybridization conditions to a nucleic acid molecule having the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof. Preferably, the polypeptides or proteins also exhibit at least one structural and/or functional feature of a polypeptide of the invention.

Also within the invention are polypeptides which are naturally occurring allelic variants of a polypeptide that includes the amino acid sequence of a protein coded for by any nucleic acid sequence recited or referred to by accession number in Table I, wherein the polypeptide is encoded by a nucleic acid molecule which hybridizes to a nucleic acid molecule having the sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof under stringent conditions.

The invention also features nucleic acid molecules that hybridize under stringent conditions to a nucleic acid molecule having the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof. In one embodiment, the nucleic acid molecules are at least 50, 100, 150, 200, 272, 300, 400, 500, 550, 600, 650, 700, 750, 800, 1000, 1100, 1200, 1300, 1400, 1500, 1600, 1600, 1700, 1800, 1900, 2000, 2100, 2200, 2300, 2400, 2500, 2600 or 2700 nucleotides in length and hybridize under stringent conditions to a nucleic acid molecule comprising the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof.

The invention also features nucleic acid molecules at least 15, preferably 50, 75, 100, 150, 200, 250, 300, 350, 400, 500, 600 or more nucleotides in length and hybridize under stringent conditions to a nucleic acid molecule comprising the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I, or a complement thereof. Preferably, such nucleic acid molecules encode polypeptides or proteins that exhibit at least one structural and/or functional feature of a polypeptide of the invention.

In one embodiment, the invention provides an isolated nucleic acid molecule which is antisense to the coding strand of a nucleic acid of the invention.

As used herein, the term “nucleic acid molecule” is intended to include DNA molecules (e.g., cDNA or genomic DNA) and RNA molecules (e.g., mRNA) and analogs of the DNA or RNA generated using nucleotide analogs. The nucleic acid molecule can be single-stranded or double-stranded, but preferably is double-stranded DNA.

An “isolated” nucleic acid molecule is one which is separated from other nucleic acid molecules which are present in the natural source of the nucleic acid molecule. Preferably, an “isolated” nucleic acid molecule is free of sequences (preferably protein encoding sequences) which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kB, 4 kB, 3 kB, 2 kB, 1 kB, 0.5 kB or 0.1 kB of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. As used herein, the term “isolated” when referring to a nucleic acid molecule does not include an isolated chromosome.

In instances wherein the nucleic acid molecule is a cDNA or RNA, e.g., mRNA, molecule, such molecules can include a poly A “tail”, or, alternatively, can lack such a 3′ tail. Although cDNA or RNA nucleotide sequences may be depicted herein with such tail sequences, it is to be understood that cDNA nucleic acid molecules of the invention are also intended to include such sequences lacking the depicted poly A tails.

A nucleic acid molecule of the present invention, e.g., a nucleic acid molecule having the nucleotide sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I or a complement thereof, can be isolated using standard molecular biology techniques and the sequence information provided herein. Using all or a portion of the nucleic acid sequences of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I as a hybridization probe, nucleic acid molecules of the invention can be isolated using standard hybridization and cloning techniques (e.g., as described in Sambrook et al., eds., Molecular Cloning: A Laboratory Manual, 2nd ed., Cold Spring Harbor Laboratory, Cold Spring Harbor Laboratory Press, Cold Spring Harbor, N.Y., 1989).

A nucleic acid molecule of the invention can be amplified using cDNA, mRNA or genomic DNA as a template and appropriate oligonucleotide primers according to standard PCR amplification techniques. The nucleic acid so amplified can be cloned into an appropriate vector and characterized by DNA sequence analysis. Furthermore, oligonucleotides corresponding to all or a portion of a nucleic acid molecule of the invention can be prepared by standard synthetic techniques, e.g., using an automated DNA synthesizer.

As used herein, the term “hybridizes under stringent conditions” is intended to describe conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. A preferred, non-limiting example of stringent hybridization conditions are hybridization in 6× sodium chloride/sodium citrate (SSC) at about 45° C., followed by one or more washes in 0.2× SSC, 0.1% SDS at 50-65° C. Preferably, an isolated nucleic acid molecule of the invention that hybridizes under stringent conditions to the sequence of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; SEQ ID NO: 75; or a sequence referred to by accession number for any ERM listed in table I or a complement thereof, corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In addition to naturally-occurring allelic variants of a nucleic acid molecule of the invention sequence that may exist in the population, the skilled artisan will further appreciate that changes can be introduced by mutation thereby leading to changes in the amino acid sequence of the encoded protein, without altering the biological activity of the protein. For example, one can make nucleotide substitutions leading to amino acid substitutions at “non-essential” amino acid residues. A “non-essential” amino acid residue is a residue that can be altered from the wild-type sequence without altering the biological activity, whereas an “essential” amino acid residue is required for biological activity. For example, amino acid residues that are not conserved or only semi-conserved among homologues of various species may be non-essential for activity and thus would be likely targets for alteration. Alternatively, amino acid residues that are conserved among the homologues of various species (e.g., mouse and human) may be essential for activity and thus would not be likely targets for alteration.

Another aspect of the invention provides vectors, e.g., recombinant expression vectors, comprising a nucleic acid molecule of the invention. In another embodiment, the invention provides host cells containing such a vector or engineered to contain and/or express a nucleic acid molecule of the invention. The invention also provides methods for producing a polypeptide of the invention by culturing, in a suitable medium, a host cell of the invention such that a polypeptide of the invention is produced. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors, expression vectors, are capable of directing the expression of genes to which they are operably linked. In general, expression vectors of utility in recombinant DNA techniques are often in the form of plasmids (vectors). However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell. This means that the recombinant expression vectors include one or more regulatory sequences, selected on the basis of the host cells to be used for expression, which is operably linked to the nucleic acid sequence to be expressed. Within a recombinant expression vector, “operably linked” is intended to mean that the nucleotide sequence of interest is linked to the regulatory sequence(s) in a manner which allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein.

The recombinant expression vectors of the invention can be designed for expression of a polypeptide of the invention in prokaryotic (e.g., E. coli) or eukaryotic cells (e.g., insect cells (using baculovirus expression vectors), yeast cells or mammalian cells). Suitable host cells are discussed further in Goeddel, supra. Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: 1) to increase expression of recombinant protein; 2) to increase the solubility of the recombinant protein; and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein.

Examples of suitable inducible non-fusion E. coli expression vectors include pTrc (Amann et al., (1988) Gene 69:301-315) and pET 11d (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains BL21(DE3) or HMS174(DE3) from a resident λ prophage harboring a T7 gn1 gene under the transcriptional control of the lacUV 5 promoter.

One strategy to maximize recombinant protein expression in E. coli is to express the protein in a host bacteria with an impaired capacity to proteolytically cleave the recombinant protein (Gottesman, Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E. coli (Wada et al. (1992) Nucleic Acids Res. 20:2111-2118). Such alteration of nucleic acid sequences of the invention can be carried out by standard DNA synthesis techniques.

In another embodiment, the expression vector is a yeast expression vector. Examples of vectors for expression in yeast S. cerivisae include pYepSec1 (Baldari et al. (1987) EMBO J. 6:229-234), pMFa (Kurjan and Herskowitz, (1982) Cell 30:933-943), pJRY88 (Schultz et al. (1987) Gene 54:113-123), pYES2 (Invitrogen Corporation, San Diego, Calif.), and pPicZ (Invitrogen Corp, San Diego, Calif.).

Alternatively, the expression vector is a baculovirus expression vector. Baculovirus vectors available for expression of proteins in cultured insect cells (e.g., Sf 9 cells) include the pAc series (Smith et al. (1983) Mol. Cell Biol. 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al. (1987) EMBO J. 6:187-195). When used in mammalian cells, the expression vector's control functions are often provided by viral regulatory elements. For example, commonly used promoters are derived from polyoma, Adenovirus 2, cytomegalovirus and Simian Virus 40. For other suitable expression systems for both prokaryotic and eukaryotic cells see chapters 16 and 17 of Sambrook et al., supra.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert et al. (1987) Genes Dev. 1:268-277), lymphoid-specific promoters (Calame and Eaton (1988) Adv. Immunol. 43:235-275), in particular promoters of T cell receptors (Winoto and Baltimore (1989) EMBO J. 8:729-733) and immunoglobulins (Banerji et al. (1983) Cell 33:729-740; Queen and Baltimore (1983) Cell 33:741-748), neuron-specific promoters (e.g., the neurofilament promoter; Byrne and Ruddle (1989) Proc. Natl. Acad. Sci. USA 86:5473-5477), pancreas-specific promoters (Edlund et al. (1985) Science 230:912-916), and mammary gland-specific promoters (e.g., milk whey promoter; U.S. Pat. No. 4,873,316 and European Application Publication No. 264,166). Developmentally-regulated promoters are also encompassed, for example the mouse hox promoters (Kessel and Gruss (1990) Science 249:374-379) and the beta-fetoprotein promoter (Campes and Tilghman (1989) Genes Dev. 3:537-546).

The invention further provides a recombinant expression vector comprising a DNA molecule of the invention cloned into the expression vector in an antisense orientation. That is, the DNA molecule is operably linked to a regulatory sequence in a manner which allows for expression (by transcription of the DNA molecule) of an RNA molecule which is antisense to the mRNA encoding a polypeptide of the invention. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

Another aspect of the invention pertains to host cells into which a recombinant expression vector of the invention has been introduced. The terms “host cell” and “recombinant host cell” are used interchangeably herein. It is understood that such terms refer not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic (e.g., E. coli) or eukaryotic cell (e.g., insect cells, yeast or mammalian cells).

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation. Suitable methods for transforming or transfecting host cells can be found in Sambrook, et al. (supra), and other laboratory manuals.

Another aspect of this invention features isolated or recombinant proteins and polypeptides of the invention. Preferred proteins and polypeptides possess at least one biological activity possessed by the corresponding naturally-occurring human polypeptide. An activity, a biological activity, or a functional activity of a polypeptide or nucleic acid of the invention refers to an activity exerted by a protein, polypeptide or nucleic acid molecule of the invention on a responsive cell as determined in vivo or in vitro, according to standard techniques. Such activities can be a direct activity, such as an association with or an enzymatic activity on a second protein, or an indirect activity, such as a cellular signaling activity mediated by interaction of the protein with a second protein.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

To determine the percent identity of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (, % identity=# of identical positions/total # of positions (e.g., overlapping positions)×100). In one embodiment, the two sequences are the same length. The determination of percent identity between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad. Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to a nucleic acid molecules of the invention. BLAST protein searches can be performed with the XBLAST program, score=50, wordlength=3 to obtain amino acid sequences homologous to a protein molecules of the invention. To obtain gapped alignments for comparison purposes, Gapped BLAST can be utilized as described in Altschul et al. (1997) Nucleic Acids Res. 25:3389-3402. Alternatively, PSI-Blast can be used to perform an iterated search which detects distant relationships between molecules (Id.). When utilizing BLAST, Gapped BLAST, and PSI-Blast programs, the default parameters of the respective programs (e.g., XBLAST and NBLAST) can be used. See http://www.ncbi.nlm.nih.gov. Another preferred, non-limiting example of a mathematical algorithm utilized for the comparison of sequences is the algorithm of Myers and Miller, CABIOS (1989). Such an algorithm is incorporated into the ALIGN program (version 2.0) which is part of the CGC sequence alignment software package. When utilizing the ALIGN program for comparing amino acid sequences, a PAM120 weight residue table, a gap length penalty of 12, and a gap penalty of 4 can be used. Additional algorithms for sequence analysis are known in the art and include ADVANCE and ADAM as described in Torellis and Robotti (1994) Comput. Appl. Biosci., 10:3-5; and FASTA described in Pearson and Lipman (1988) Proc. Natl. Acad. Sci. 85:2444-8. Within FASTA, ktup is a control option that sets the sensitivity and speed of the search. If ktup=2, similar regions in the two sequences being compared are found by looking at pairs of aligned residues; if ktup=1, single aligned amino acids are examined. ktup can be set to 2 or 1 for protein sequences, or from 1 to 6 for DNA sequences. The default if ktup is not specified is 2 for proteins and 6 for DNA. For a further description of FASTA parameters, see http://bioweb.pasteur.fr/docs/man/man/fasta.1.html#sect2, the contents of which are incorporated herein by reference.

The percent identity between two sequences can be determined using techniques similar to those described above, with or without allowing gaps. In calculating percent identity, typically exact matches are counted.

The invention also provides chimeric or fusion proteins. As used herein, a “chimeric protein” or “fusion protein” comprises all or part (preferably biologically active) of a polypeptide of the invention operably linked to a heterologous polypeptide (i.e., a polypeptide other than the same polypeptide of the invention). Within the fusion protein, the term “operably linked” is intended to indicate that the polypeptide of the invention and the heterologous polypeptide are fused in-frame to each other. The heterologous polypeptide can be fused to the N-terminus or C-terminus of the polypeptide of the invention.

One useful fusion protein is a GST fusion protein in which the polypeptide of the invention is fused to the C-terminus of GST sequences. Such fusion proteins can facilitate the purification of a recombinant polypeptide of the invention.

In another embodiment, the fusion protein contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

The invention further features antibodies, such as monoclonal or polyclonal antibodies or fragments thereof, that specifically bind a polypeptide of the invention. The antibodies of the invention can be conjugated antibodies comprising, for example, therapeutic or diagnostic agents. For example, the antibodies can be conjugated to a therapeutic moiety such as a chemotherapeutic cytotoxin, e.g., a cytostatic or cytocidal agent (e.g., paclitaxol, cytochalasin B or diphtheria toxin), a thrombotic or anti-angiogenic agent or a radioactive or fluorescent label.

An isolated polypeptide of the invention, or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of a protein of the invention, and encompasses an epitope of the protein such that an antibody raised against the peptide forms a specific immune complex with the protein.

Preferred epitopes encompassed by the antigenic peptide are regions that are located on the surface of the protein, e.g., hydrophilic regions.

An immunogen typically is used to prepare antibodies by immunizing a suitable subject, (e.g., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention. A molecule which specifically binds to a given polypeptide of the invention is a molecule which binds the polypeptide, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies. The term “monoclonal antibody” or “monoclonal antibody composition”, as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. The antibody titer in the immunized subject can be monitored over time by standard techniques, such as with an enzyme linked immunosorbent assay (ELISA) using immobilized polypeptide. If desired, the antibody molecules can be isolated from the mammal (e.g., from the blood) and further purified by well-known techniques, such as protein A chromatography to obtain the IgG fraction. At an appropriate time after immunization, e.g., when the specific antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Hybridoma cells producing a monoclonal antibody of the invention are detected by screening the hybridoma culture supernatants for antibodies that bind the polypeptide of interest, e.g., using a standard ELISA assay.

Alternative to preparing monoclonal antibody-secreting hybridomas, a monoclonal antibody directed against a polypeptide of the invention can be identified and isolated by screening a recombinant combinatorial immunoglobulin library (e.g., an antibody phage display library) with the polypeptide of interest. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

Additionally, recombinant antibodies, such as chimeric and humanized monoclonal antibodies, comprising both human and non-human portions, which can be made using standard recombinant DNA techniques, are within the scope of the invention. A chimeric antibody is a molecule in which different portions are derived from different animal species, such as those having a variable region derived from a murine mAb and a human immunoglobulin constant region. (See, e.g., Cabilly et al., U.S. Pat. No. 4,816,567; and Boss et al., U.S. Pat. No. 4,816397, which are incorporated herein by reference in their entirety.) Humanized antibodies are antibody molecules from non-human species having one or more complementarily determining regions (CDRs) from the non-human species and a framework region from a human immunoglobulin molecule. (See, e.g., Queen, U.S. Pat. No. 5,585,089, which is incorporated herein by reference in its entirety.) Such chimeric and humanized monoclonal antibodies can be produced by recombinant DNA techniques known in the art, for example using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Canc. Res. 47:999-1005; Wood et al. (1985) Nature 314:446-449; and Shaw et al. (1988) J. Natl. Cancer Inst. 80:1553-1559); Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060.

In addition, the polypeptides of the invention or biologically active portions thereof, or antibodies of the invention, can be incorporated into pharmaceutical compositions, which optionally include pharmaceutically acceptable carriers.

In another aspect, the present invention provides methods for detecting the presence, activity or expression of a polypeptide of the invention in a biological sample by contacting the biological sample with an agent capable of detecting an indicator of the presence, activity or expression such that the presence activity or expression of a polypeptide of the invention is detected in the biological sample.

In another aspect, the invention provides methods for modulating activity of a polypeptide of the invention comprising contacting a cell with an agent that modulates (inhibits or stimulates) the activity or expression of a polypeptide of the invention such that activity or expression in the cell is modulated. In one embodiment, the agent is an antibody that specifically binds to a polypeptide of the invention. In another embodiment, the agent is a fragment of a polypeptide of the invention or a nucleic acid molecule encoding such a polypeptide fragment.

In another embodiment, the agent modulates expression of a polypeptide of the invention by modulating transcription, splicing, or translation of an mRNA encoding a polypeptide of the invention. In yet another embodiment, the agent is a nucleic acid molecule having a nucleotide sequence that is antisense to the coding strand of an mRNA encoding a polypeptide of the invention.

The present invention also provides methods to treat a subject having a disorder characterized by aberrant activity of a polypeptide of the invention or aberrant expression of a nucleic acid of the invention by administering an agent which is a modulator of the activity of a polypeptide of the invention or a modulator of the expression of a nucleic acid of the invention to the subject. In one embodiment, the modulator is a protein of the invention. In another embodiment, the modulator is a nucleic acid of the invention. In other embodiments, the modulator is a peptide, peptidomimetic, or other small molecule.

The present invention also provides diagnostic assays for identifying the presence or absence of a genetic lesion or mutation characterized by at least one of: (i) aberrant modification or mutation of a gene encoding a polypeptide of the invention, (ii) mis-regulation of a gene encoding a polypeptide of the invention, and (iii) aberrant post-translational modification of the invention wherein a wild-type form of the gene encodes a protein having the activity of the polypeptide of the invention.

In another aspect, the invention provides a method for identifying a compound that binds to or modulates the activity of a polypeptide of the invention. In general, such methods entail measuring a biological activity of the polypeptide in the presence and absence of a test compound and identifying those compounds which alter the activity of the polypeptide.

The invention also features methods for identifying a compound which modulates the expression of a polypeptide or nucleic acid of the invention by measuring the expression of the polypeptide or nucleic acid in the presence and absence of the compound.

For all nucleic acids of the invention, in a preferred embodiment, the nucleic acids of the invention are any or all those referred to above, with the proviso that the sequence of any given nucleic acid of the invention does not comprise or is not 100% identical to the sequence provided in table I, or any sequence referred to by accession number or name in table I.

Pharmaceutical Compositions

The SERMs identified by the methods of the invention, nucleic acid molecules, polypeptides, and antibodies (also referred to herein as “active compounds”) of the invention can be incorporated into pharmaceutical compositions suitable for administration. Such compositions typically comprise the SERM, nucleic acid molecule, protein, or antibody and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The invention includes methods for preparing pharmaceutical compositions for modulating the expression or activity of a polypeptide or nucleic acid of the invention. Such methods comprise formulating a pharmaceutically acceptable carrier with an agent which modulates expression or activity of a polypeptide or nucleic acid of the invention. Such compositions can further include additional active agents. Thus, the invention further includes methods for preparing a pharmaceutical composition by formulating a pharmaceutically acceptable carrier with an agent which modulates expression or activity of a polypeptide or nucleic acid of the invention and one or more additional active compounds.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersions. For intravenous administration, suitable carriers include physiological saline, bacteriostatic water, Cremophor EL™ (BASF; Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a polypeptide or antibody) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

For antibodies, the preferred dosage is 0.1 mg/kg to 100 mg/kg of body weight (generally 10 mg/kg to 20 mg/kg). If the antibody is to act in the brain, a dosage of 50 mg/kg to 100 mg/kg is usually appropriate. Generally, partially human antibodies and fully human antibodies have a longer half-life within the human body than other antibodies. Accordingly, lower dosages and less frequent administration is often possible. Modifications such as lipidation can be used to stabilize antibodies and to enhance uptake and tissue penetration (e.g., into the brain). A method for lipidation of antibodies is described by Cruikshank et al. ((1997) J. Acquired Immune Deficiency Syndromes and Human Retrovirology 14:193).

As defined herein, a therapeutically effective amount of a SERM, protein or polypeptide (i.e., an effective dosage) ranges from about 0.001 to 30 mg/kg body weight, preferably about 0.01 to 25 mg/kg body weight, more preferably about 0.1 to 20 mg/kg body weight, and even more preferably about 1 to 10 mg/kg, 2 to 9 mg/kg, 3 to 8 mg/kg, 4 to 7 mg/kg, or 5 to 6 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a protein, polypeptide, or antibody can include a single treatment or, preferably, can include a series of treatments. In a preferred example, a subject is treated with a SERM, antibody, protein, or polypeptide in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of antibody, protein, or polypeptide used for treatment may increase or decrease over the course of a particular treatment. Changes in dosage may result and become apparent from the results of diagnostic assays as described herein.

The present invention encompasses agents which modulate expression or activity. An agent may, for example, be a small molecule. For example, such small molecules include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

It is understood that appropriate doses of small molecule agents depends upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram. It is furthermore understood that appropriate doses of a small molecule depend upon the potency of the small molecule with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The nucleic acid molecules of the invention can be inserted into vectors and used as gene therapy vectors. Gene therapy vectors can be delivered to a subject by, for example, intravenous injection, local administration (U.S. Pat. No. 5,328,470) or by stereotactic injection (see, e.g., Chen et al. (1994) Proc. Natl. Acad. Sci. USA 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system.

The pharmaceutical compositions can be included in a container, pack, or dispenser together with instructions for administration.

Methods of Treatment

The present invention provides for both prophylactic and therapeutic methods of treating a subject at risk of (or susceptible to) a disorder or having a disorder associated with aberrant expression or activity of a polypeptide of the invention. The nucleic acids, polypeptides, and modulators thereof of the invention can be used to treat cancer (e.g., breast or endometrial), osteoporosis and cardiovascular disease (e.g., myocardial infarction, atherosclerosis), restenosis, Alzheimer's disease, urinary incontinence, hair loss and decreased or absent libido, as well as other disorders described herein. SERMs and other therapeutic agents may be administered to a patient in an amount sufficient to exert a therapeutic effect. Such an effect may prevent onset of a disease, or may delay disease onset or reduce disease severity or duration. Conditions that may benefit from such therapies (prophylactically or after onset of the disease) include cancer (e.g., breast or endometrial), osteoporosis and cardiovascular disease (e.g., myocardial infarction, atherosclerosis), restenosis, Alzheimer's disease, urinary incontinence, hair loss and decreased or absent libido

Prophylactic Methods

In one aspect, the invention provides a method for preventing in a subject, a disease or condition associated with an aberrant expression or activity of a polypeptide of the invention, by administering to the subject an agent which modulates expression or at least one activity of the polypeptide. Subjects at risk for a disease which is caused or contributed to by aberrant expression or activity of a polypeptide of the invention can be identified by, for example, any or a combination of diagnostic or prognostic assays as described herein. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

Therapeutic Methods

Another aspect of the invention pertains to methods of modulating expression or activity of a polypeptide of the invention for therapeutic purposes. The modulatory method of the invention involves contacting a cell with an agent that modulates one or more of the activities of the polypeptide. An agent that modulates activity can be an agent as described herein, such as a nucleic acid or a protein, a naturally-occurring cognate ligand of the polypeptide, a peptide, a peptidomimetic, or other small molecule. In one embodiment, the agent stimulates one or more of the biological activities of the polypeptide. Examples of such stimulatory agents include the active polypeptide of the invention and a nucleic acid molecule encoding the polypeptide of the invention that has been introduced into the cell. In another embodiment, the agent inhibits one or more of the biological activities of the polypeptide of the invention. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the present invention provides methods of treating an individual afflicted with a disease or disorder characterized by aberrant expression or activity of a polypeptide of the invention. In one embodiment, the method involves administering an agent (e.g., an agent identified by a screening assay described herein), or combination of agents that modulates (e.g., upregulates or downregulates) expression or activity. In another embodiment, the method involves administering a polypeptide of the invention or a nucleic acid molecule of the invention as therapy to compensate for reduced or aberrant expression or activity of the polypeptide.

Stimulation of activity is desirable in situations in which activity or expression is abnormally low or downregulated and/or in which increased activity is likely to have a beneficial effect. Conversely, inhibition of activity is desirable in situations in which activity or expression is abnormally high or upregulated and/or in which decreased activity is likely to have a beneficial effect.

This invention is further illustrated by the following examples which should not be construed as limiting. The contents of all references, patents and published patent applications cited throughout this application are hereby incorporated by reference.

### EXAMPLES

### Example 1

Preparation of Estrogen-Receptor Enhanced Vascular Endothelial Cells

Estrogen receptor α (ERα)-enhanced vascular endothelial cells were prepared by infecting primary vascular endothelial cells with a retrovirus expressing human papillomavirus E6/E7, which leads to cellular immortalization. The retrovirus expression vector LXSN16E6E7 contains the immortalizing oncogenes E6 and E7 from human papilloma virus type 16 and a neomycin resistance gene. (Halbert, C. L., Demers, F. W., and Galloway, D. A. 1991. J Virol. 65: 473-478 (1)). This vector was transfected into PA317 cells, an amphotrophic packaging cell line, by calcium phosphate precipitation. Stably clonal lines were established by G418 selection. A negative control line expressing the empty retrovirus LXSN was established. Viruses produced by the clonal lines of PA317 were used to infect second passage primary human aortic endothelial cells obtained from Cell Applications, Inc., San Diego, Calif. Following infection and selection with G418, the LXSN infected cells ceased growth after approximately 7 passages where the LXSN16E6E7 infected cells were growth extended beyond 20 passages.

These immortalized cells showed very low expression of ERβ mRNA and no detectable ERα mRNA, and no estrogen response could be detected. The cells expressed the expected endothelial markers. The levels of ERα were increased by infection of the cells with a retrovirus that expresses recombinant ERα (Green, S. et al, Nature 1986 320: 134-137). Retroviral vectors were constructed to express estrogen receptor α(LXSZERα) or β (LXSZERβ) and confer zeocin resistance. A clonal line of the immortalized vascular endothelial cells at passage 8 post-immortalization was infected with virus produced by PA317 cell transfected with these ER expressing retroviruses. Following zeocin treatment and clonal selection, one immortalized vascular endothelial cell line expressing ERα and one expressing ERβ were established. Estrogen receptor expression was verified by RNase protection, Western blotting, and immunostaining.

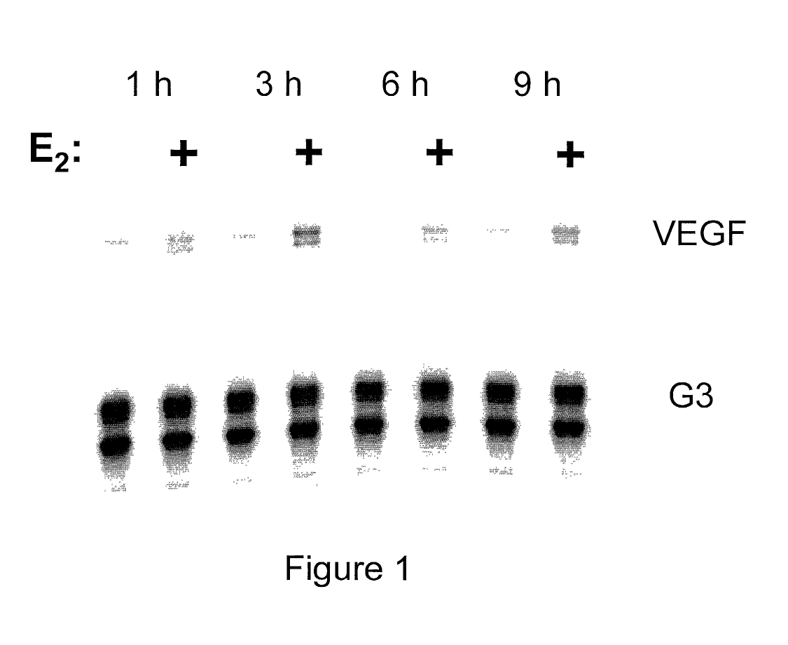

Enhancement of the level of ERα protein in these cells was found in greater than 90% of the cells by immunocytochemistry. The expression of vascular endothelial growth factor (VEGF) mRNA was increased three fold by treating the cell line (VE-ERα) with estrogen (shown in FIG. 1 by, RNAse protection analysis). This demonstrates the estrogen responsiveness of these cells.

### Example 2

Identification of Estrogen-Regulated Markers

This Example illustrates the use of VE-ERα cells to screen for estrogen-regulated markers.

The Incyte UniGEM technology was used to survey a large panel of genes and to establish the estrogen regulated gene profile of the VE-ERα cells. VE-ERα cells were treated for 6 hours with 17-β estradiol or vehicle control (ethanol). Poly (A) RNA was isolated and this RNA was used to synthesize Cy3 or Cy5 labeled cDNA for simultaneous probing of Incyte UniGem V. Reciprocal labeling was done and two UniGems were probed (for one GEM the estradiol probe was labeled with Cy3 and ethanol probe with Cy5 and this labeling was reversed for the second GEM). UniGem V contains approximately 8500 human sequences including both known genes and ESTs. More than 20 genes were identified which were regulated more than 2 fold on both UniGEMs (Table I, indicated as UniGem. The sequence indicated in Table I with RF were identified by RNA fingerprinting, a more stringent version of differential display. RNA fingerprinting is based upon the use of arbitrary primers and primer combinations to carry out PCR amplifications from cDNA populations derived from cells or tissues of interest. The process generates a series of radioactively-labeled fragments which can be resolved by PAGE and visualized by autoradiography. (Mathieu-Daude, et al. Methods Enzymol 1999;303:309; Stone, et al., Nucleic Acids Res 1994 Jul 11;22(13):2612-8.)

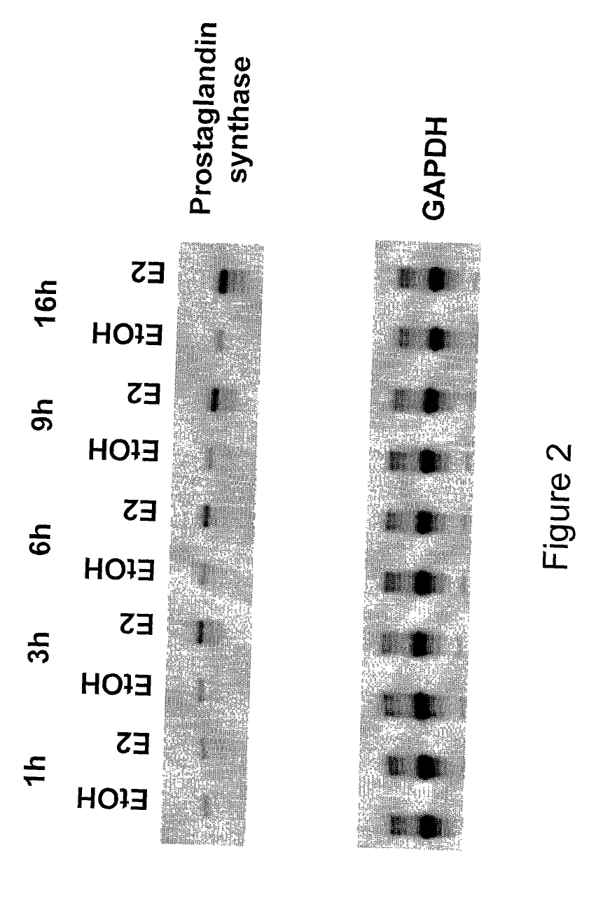

Side by side comparisons of such gel displays generated from distinct cell states or tissues types allow the investigator to identify differentially gene expression in the target cells or tissues. RNA was isolated from VE103ERa cells (control Vs treated for 24 hours with 100 nM 17-β-estradiol). For initial confirmation studies of genes indentified by RNA fingerprinting, a northern blot containing poly (A) RNA (vehicle control versus 100 nM 17-β-estradiol) is probed with each gene. This step is not necessary with genes identified by the UniGem where an estrogen treatment time course was performed. RNA was isolated and RNase protection analysis was used to determine estrogen responses (FIG. 2). All 20 of the genes examined were confirmed to be regulated to a higher fold modulation at 24 hours than the 6 hours used for the UniGem experiment. Table I provides a summary of estrogen-regulated markers identified in this study.

TABLE I
Representative Estrogen-Regulated Markers

| | Sequence or Data- | | |
|---|---|---|---|
| ERM | base Name | Acc. No. | Detection |
| 1 | Tumor necrosis re- | M32315 | UniGem |
| | ceptor 2 (75kD) | | |
| 2 | Selectin P (granule | M25322 | UniGem |
| | membrane protein 140) | | |
| 3 | Prostaglandin I2 | D84124 | UniGem&RF |
| | (prostacyclin) syn- | | |
| | thase The enzyme | | |
| | required for the | | |
| | last step in the | | |
| | synthesis of PGI2, | | |
| | an inhibitor of | | |
| | atherogenesis | | |
| 4 | Latent transforming | M34057 | UniGem&RF |
| | growth factor beta | | |
| | binding protein | | |
| 5 | N-acetyllactosa- | D29805 | UniGem&RF |
| | mine synthase (-ga- | | |
| | lactosyl transfe- | | |
| | rase) | | |
| 6 | Iduronate 2-sulfa- | L13329 | UniGem |
| | tase (Hunter syn- | | |
| | drome) | | |
| 7 | RIG mRNA (Regulated I | U32331 | UniGem |
| | n Glioma) | | |
| 8 | NEF-associated | D30755 | UniGem |
| | factor 1 alpha | | |
| 9 | hbc647 | U68494 | UniGem |
| 10 | Follistatin | M19481 | UniGem |
| 11 | Epidermal growth | U12535 | UniGem |
| | factor receptor | | |
| | kinase substrate | | |
| | (eps8) A signaling | | |
| | molecule that | | |
| | serves as a sub- | | |
| | strate for receptor | | |
| | and nonreceptor ty- | | |
| | rosine kinases. | | |
| | Eps8 appears to be | | |
| | involved in cell | | |
| | proliferation and | | |
| | cytoskeleton remo- | | |
| | deling. | | |
| 12 | Cyclin A1 A cyclin | U66838 | UniGem |
| | A-like gene, hav- | | |
| | ing 48% identity | | |
| | to human cyclin A | | |
| | and 84% identity to | | |
| | murine cyclin A1 | | |
| | (Yang et al., | | |
| | Cancer Res. 57:913- | | |
| | 920, 1997) | | |
| 13 | Serum inducible kinase mRNA | AF059617 | UniGem |
| 14 | Sarc/endoplasmic | Y15724 | UniGem |
| | reticulum CaATPase | | |
| 15 | Podocalyxin protein | U97519 | UniGem |
| | A ligand for L-se- | | |
| | lectin similar in | | |
| | structure to CD34 | | |
| 16 | Natriuretic pep- | L13436 | UniGem |
| | tide receptor B | | |
| | (NPR-B) The guany- | | |
| | lyl cyclase recep- | | |
| | tor for C-type na- | | |
| | triuretic peptide | | |
| | (CNP), and endothe- | | |
| | lium dependent ven- | | |
| | odilator that has | | |
| | antimitogenic acti- | | |
| | vity on smooth mu- | | |
| | scle cells | | |
| 17 | Leupaxin | AF062075 | UniGem |
| 18 | Ezrin-radixin-moe- | AF015926 | UniGem |
| | sin binding phos- | | |
| | phoprotein | | |
| 19 | Hexokinase I | AF016365 | UniGem |
| 20 | Connexin 37 (Gap | M96789 | UniGem |
| | junction protein | | |
| | alpha 4) A gap | | |
| | junction protein in | | |
| | endothelial cells | | |
| 21 | Diastrophic dyspla- | U14528 | UniGem |
| | sia sulphate trans- | | |
| | porter This gene is | | |
| | mutated in a form | | |
| | of osteochondrodys- | | |
| | plasia that leads | | |
| | to dwarfism and | | |
| | spinal deformation | | |
| 22 | Cadherin 11(OB-cad- | D21255 | UniGem |
| | herin) | | |
| 23 | 6 phospho fructo-2 | D49817 | UniGem |
| | kinase/fructose 2,6 | | |
| | bis phosphatase | | |
| 24 | Ras-related rhoB | X06820 | RF |
| | A member of the Rho | | |
| | family of GTPases | | |
| | that includes Rac, | | |
| | Rho and Cdc42. Rho | | |
| | B has been implica- | | |
| | ted in cell growth | | |
| | control, actin re- | | |
| | gulation and adhe- | | |
| | sion dependent via- | | |
| | bility | | |
| 25 | Transglutaminase | M55153.1 | RF |
| 26 | Crk-associated sub- | AB040024 | RF |
| | strate p130Cas A | | |
| | signaling molecule | | |
| | important for path- | | |
| | ways involved in | | |
| | cell invasion, ad- | | |
| | hesion and survival | | |
| 27 | Clusterin (Apolipo- | J02908 | RF |
| | protein J) Cluste- | | |
| | rin has been found | | |
| | at increased levels | | |
| | in atherosclerotic | | |
| | lesions and in | | |
| | wound repair. | | |
| 28 | Connective tissue | U14750 | RF |
| | growth factor Medi- | | |
| | ates cell adhesion | | |
| | and migration | | |
| 29 | Putative type II | U14750 | RF |
| | membrane protein | | |
| 30 | Legumain | Y09862 | RF |
| 31 | Complement compo- | K02403 | RF |
| | nent C4A | | |
| 32 | WDR1 protein | AF020056 | RF |
| 33 | ADP ribosylation | AF052179 | RF |
| | factor | | |
| 34 | Casein Kinase II | M55265 | RF |
| | (alpha) | | |
| 35 | CPSF (cleavage and | AF171877 | RF |
| | polyadenylation | | |
| | specificity factor) | | |
| 36 | ATP Sulfurylase/APS | AF074331 | RF |
| | Kinase 2 (PAPS syn- | | |
| | thetase 2) A bi- | | |
| | functional protein | | |
| | used to synthesize | | |
| | the universal sul- | | |
| | fate donor PAPS (3′ | | |
| | phosphoadenosine 5′ | | |
| | phosphosulfate). A | | |
| | mutation of this | | |
| | gene leads to a | | |
| | cartilage/skeletal | | |
| | disease (spondylo- | | |
| | epimetaphyseal dys- | | |
| | plasia | | |
| 37 | Human ubiquitin | U45328 | RF |
| | conjugating enzyme | | |
| | type UBC9 | | |
| 38 | Small membrane pro- | AF081282 | RF |
| | tein 1 (SMP1) mRNA | | |
| 39 | Casein kinase II | M30448 | RF |
| | (beta) | | |
| 40 | Trabeculin-alpha | AE141968 | RF |
| | mRNA; actin binding | | |
| | protein ABP62O | | |
| 41 | Ca+ Dependent Pro- | X04106 | RF |
| | tease (Homo sapiens | | |
| | calpain, small po- | | |
| | lypeptide (CAPN4) | | |
| 42 | Transforming acidic | AF093543 | RF |
| | coiled-coil con- | | |
| | taining protein 3 | | |
| | (TACC3) mRNA | | |
| 43 | Sterile-alpha motif | AF251441 | RF |
| | and leucine zipper | | |
| | containing kinase | | |
| | (AZK), mixed li- | | |
| | neage kinase | | |
| 44 | Putative TH1 pro- | AJ238379 | RF |
| | tein | | |
| 45 | 60S acidic ribosomal | AF173378 | RF |
| | protein PO mRNA) | | |
| 46 | Tubulin, beta,2 | NM006088 | RF |
| | (TUBB2) mRNA | | |
| 47 | HLA-E gene (major | X64879 | RF |
| | histocompatibility | | |
| | complex) | | |
| 48 | Myosin X (MYO10) | AF234532 | RF |
| | mRNA | | |
| 49 | VE-cadherin-5 A me- | X79981 | RF |
| | diator of endothe- | | |
| | lial cell adhesion | | |
| 50 | Bromodomain adja- | AB002312 | RF |
| | cent to zinc finger | | |
| | domain, 2A (BAZ2A) | | |
| 51 | Carbohydrate (chon- | AE083066 | RF |
| | droitin6/keratan) | | |
| | sulfotransferase 2 | | |
| | The key enzyme in | | |
| | the biosynthesis of | | |
| | chondroitin 6-sul- | | |
| | fate, a glycoamino- | | |
| | glylcan implicated | | |
| | in chondrogenesis, | | |
| | neoplasia, athero- | | |
| | sclerosis and other | | |
| | processes. Oversul- | | |
| | fated chondroitin | | |
| | sulfate isomers | | |
| | have anticoagulant | | |
| | and anti-athero- | | |
| | sclerotic activi- | | |
| | ties suggesting a | | |
| | pertinent role in | | |
| | the disease pro- | | |
| | cess. | | |
| 52 | Endothelin-1 | Y00749 | RF |
| 53 | ABP32 (peflin (PEF) | AB026628 | RF |
| 54 | TADA-1 protein mRNA | AF132000 | RF |
| 55 | CDC45L (CDC45L) | AF081535 | RF |
| | mRNA | | |
| 56 | Homo sapiens cos- | U82671 | RF |
| | mids | | |
| 57 | Homo sapiens full | AA747315 | UniGem |
| | length insert cDNA | | |
| | Y073E04 | | |
| | TTTTTTTTTCTTTAAATAA | | |
| | CAATTTGACAAAAGGGTGA | | |
| | AGAAATCCTAAACAAGGTA | | |
| | TTGAGGCCAGTGTCCAGGC | | |
| | TGCATTCAGTTCACAGAAC | | |
| | TGTCCTCAGGACGTTGCAT | | |
| | GGAACTGGAAATGTGTATA | | |
| | ATTACAGAAGAAAACAGGG | | |
| | AGGACTTAGTGCAGAGAGG | | |
| | AGACGAGTGTGGACGGGCA | | |
| | ACAGCATCCTTAGTCTTTC | | |
| | ATATTTATATATGGTATAT | | |
| | GTATTTTCTATATATATAT | | |
| | TTATATATTTTACATCCAG | | |
| | GTATCCCAGTCATCTGTAC | | |
| | CATTTCCCAGGGAGACATG | | |
| | GGTGCTTCCAAGGCGAGAC | | |
| | AGGAAAGGGTTAGGCAGGG | | |
| | AAGGGGCAGCGACGGTGCA | | |
| | GGCTGGGGCTTGGCTCACA | | |
| | GAAGCTGCAGGAGCTTCAG | | |
| | CAGCTGTAAGAGGGCCCCG | | |
| | GGCTCCGCAGACGCCAGGT | | |
| | ACTGAGNCAAAGCCAGTCC | | |
| | TCCAGCTCCACGCCCCGCC | | |
| | TGCGATCCACCGCCTTCTC | | |
| | CGCAAACTTCATCATCATC | | |
| | AGGGCCCGCTTCATGTCGA | | |
| | TCCAGTTGTGCAGCGTGCG | | |
| | CACA | | |
| 58 | EST | W28965 | UniGem |
| | TNNNTTTNTGTGGTNGTGC | | |
| | GGTGGGGGNGGTAGGGACA | | |
| | GTGTACAGAGTTCCAGCTC | | |
| | CTCTTTGATTCACCCCCTT | | |
| | CCCTCCTCCTCCCTTCTTT | | |
| | CCCATTGCTACACTAAAGG | | |
| | AGTCAGGGCTGTGTCCACC | | |
| | AATATGCTCTGTGACTAGC | | |
| | AGTCAACACAATTTCATAC | | |
| | CCAGCCAGTCATTCCGGAG | | |
| | NATTTGGGATGATAATGTT | | |
| | CCCATTCTCAGTTCCTCTG | | |
| | TGAGAATGTGNATGTCATT | | |
| | TGAAGCTCTGTGCCTCAGT | | |
| | TTCCATAGAAGTAAAACAA | | |
| | AAAGCAAGAATTCAGACTG | | |
| | ATCTTTTNCTTGNAAGAAT | | |
| | TCACANGTAATGGATAATG | | |
| | TACAAGAGATGTGGCTGAA | | |
| | TAAGAGAACTAGAACAANG | | |
| | ACTTGAACAATTTNCCTAT | | |
| | GCAACACTCTTTCCCATGG | | |
| | ACAAAGGTATCCCACAAAG | | |
| | TCATNCTTCATCGGAGGCT | | |
| | AGTTTGTCCAAAGAAAACT | | |
| | CACATACCAATGCAATAGT | | |
| | GACAAAAAAAAATTCCTCC | | |
| | ATGCCAATTCAGGAAACAG | | |
| | AACGCAAGTTGTGCTTAAG | | |
| | NCCATGGTNGGGTGTGGGA | | |
| | AGATGTTCCAANACCGNCA | | |
| | CCCCTTACCCTCNTCTATC | | |
| | ACTATCTNCAACCCCCNGN | | |
| | ANNGGGGCTCGNCAGTTTG | | |
| | NTAAAGGGGTTAATTACCA | | |
| | NNAGGGGGCNTGTCCCCCC | | |
| | AGNGGTCNCTTGGGGNGTG | | |
| | ANANNCNANAAAACCNCTN | | |
| | TCCATTTACCAAACAAANA | | |
| | CACNTCCCTGGGATTATTN | | |
| | GGGGGTTNCAGGCTTTTNA | | |
| | GNCANNTTCCANGCCNTNG | | |
| | NTCTGNCCCTNNTCCNNGG | | |
| | TTGGGAANCCATCCCCCCN | | |
| | GCTCCTGATTACCAATNCC | | |
| | CNAATCCCNNGGTGGGGTT | | |
| | GNATTCATCTCTCCCAAAA | | |
| | TGTTNGTTGGNTTCGGGGG | | |
| | GTNAAGANTTT | | |
| 59 | EST | AA194161 | UniGem |
| | CACAGGGTCACAGATTCAC | | |
| | CAAAGCTGAAAGGGCTGAG | | |
| | GAGCTCATGGTAGCCTGGG | | |
| | TTGACCTACTCTGGAGCAC | | |
| | GGTGTCTTCCTTCTAAACT | | |
| | GAGTGACTGTAGTACTATC | | |
| | TGTGCCTCTGATGGTAATA | | |
| | AAACTGACAAGATGTCTAA | | |
| | TTTTTTTTTAAGTAGGACC | | |
| | AAAGGAAAACAAGATTTAG | | |
| | ATAGTCTGACTTTGCTTTT | | |
| | GAACAACAGACATTGCAAG | | |
| | TCAAAATTGTTGTCAAATT | | |
| | TACATATGGTAAATGATGA | | |
| | ACTTTAAAAATGTGTCCAG | | |
| | GTGTTAGATGAGTTCATTA | | |
| | GACTCTTTTAATGCTAATG | | |
| | GCTAGTACCGTTTAAACAA | | |
| | AACAGCAGTTCTCTGCTGC | | |
| | AATATTCCCATTGACCACT | | |
| | TAAATGACCATAAGTGGTC | | |
| | ATTTAAGAACATGTTAGGG | | |
| | TTAGCCCTGATCTGAATAT | | |
| | AAAAGTGAGAAAAGGGCTA | | |
| | CAGTGCATTTCTTGGTAAC | | |
| | TTAAACTGAGTCCTGAAGT | | |
| | TATAATGATCCATTCGAGT | | |
| | TCNGTGATCCTTATTGGTC | | |
| | CTAATGGTGGTTCCCNACC | | |
| | GTATTGGTACAGATGAGCC | | |
| | ATACGTTCCCTTGGTACCA | | |
| | TGTAGACATGACTTCAGAT | | |
| | ACCCCTGAGGACCTNCCAG | | |
| | CA | | |
| 60 | P311 mRNA | U30521 | RF |
| | TCGACGCTNAGAGTGGACA | | |
| | GGGAGCGGGGCTTTTGTCT | | |
| | GTTGGTCTCCCTGGACTGA | | |
| | AGAGAGGGAGAATAGAAGC | | |
| | CCAAGACTAAGATTCTCAA | | |
| | AATGGTTTATTACCCAGAA | | |
| | CTCTTTGTCTGGGTCAGTC | | |
| | AAGAACCGTTTCCAAACAA | | |
| | GGACATGGAGGGAAGGCTT | | |
| | CCTAAGGGAAGACTTCCTG | | |
| | TCCCAAAGGAAGTGAGCCG | | |
| | CAAGAAGAACGATNAGACA | | |
| | AACGCTGCCTCCCTGACTC | | |
| | CACTGGGCAGCAGTGAACT | | |
| | CCGCTCCCCAAGAATCAGT | | |
| | TACCTCCACTTTTTTTAAT | | |
| | CGTAACACCTCCATTTGTA | | |
| | TTACATATGGTGTATGGGT | | |
| | ATTGATGAGGTCATGGTAT | | |
| | CATATATGGGATTTTTTTC | | |
| | TGTGTAAATCATCAAGTAT | | |
| | AAGAAGAAACTATGGGACT | | |
| | CTGAGCCTTGCTTTAGAGA | | |
| | ANTTACAGTGG | | |
| 61 | Homo sapiens clone | AF31760 | RF |
| | 24992 mRNA sequence | | |
| | CCGTCACTAKGCTGACCGC | | |
| | TGCAACTACCAGACTTTCT | | |
| | GTGCTGATGGAGCAGATGA | | |
| | GAGACGCTGTCGGCATTGC | | |
| | CAGCCTGGSAWTTTCCGAT | | |
| | GCCGGGACRAGAAGTGCGT | | |
| | GTATGAGACRTGGGTGTGC | | |
| | GAWGGGCACCCAAACTGTG | | |
| | CGGACGGCARTGATSAGTG | | |
| | GGACTGCTCCTATGTTCTG | | |
| | CCCCGCAAGGTCATTACAG | | |
| | CTGCAGTCATTGGCAGCCT | | |
| | ASTGTGCGGMCTGCTCCTG | | |
| | KTCATCGCCCTGGGCTGCA | | |
| | CCTGCWWGCTCTATGCCAT | | |
| | TCGCACCCAGGAGTACAGC | | |
| | ATCTTTGCCCCCCTCTCCC | | |
| | GGATGGAGGCTGAGATTGT | | |
| | GCATCAACAGGCACCCCCT | | |
| | TCCTACGGGCAGCTCATTG | | |
| | CCCAAGGTGCCATCCCACC | | |
| | TGTAGAATACTTTCCTACA | | |
| | GAGAATCCTAATGATAACT | | |
| | CAGTGCTGGGCAACCTGCG | | |
| | TTCT | | |
| 62 | Human DNA sequence | AL049761 | RF |
| | from clone RP5- | | |
| | 863C7 on chromosome | | |
| | 20p12.3-13. | | |
| | (AL049761) | | |
| | ATCACAATCCAGTGCAAAC | | |
| | ATAATCACAAATTGCATCT | | |
| | CTGGCACATCTGGTGCTTT | | |
| | TAGCTTCTGCACAAATTCA | | |
| | ACATGGTAACCCTCACAGC | | |
| | ATTCTAGGGCATAAAAGGG | | |
| | TCAAGTACAATAAATATCC | | |
| | ACYGTAAGTGGTCATAAGG | | |
| | AAGAAAATCACCCTGCCAC | | |
| | ACAGTCAGAATGTTTTCCC | | |
| | TTCAGCTCKCCCTACCGTT | | |
| | GAGAAGCGCTATAAAAGGA | | |
| | GGCTTTAKCSTCCTCTAAA | | |
| | GTTTACCATTATATTCACT | | |
| | AAAAGCCACCACCTTGATA | | |
| | AAGTTACTAAAGCCAAGAT | | |
| | GGGTTACAAAGTTAGATAA | | |
| | GTTCATTAAATTCAACTCC | | |
| | CCAAACAATTACGTTTATC | | |
| | TATGATGCCTAGGCGAGAA | | |
| | AGGCCTGTGAATCTATAAS | | |
| | GTAGGAGAATGGGGAACTA | | |
| | AGATTTTAGGCCTTAAAAT | | |
| | ATGTTGGAA | | |
| 63 | Novel Sequence | | RF |
| | CCGTCACTAGGCTGCCAGA | | |
| | AGATCCTCCAAGGAGCCAT | | |
| | CCCAGACCCAGTGATCTGC | | |
| | TGCCACTCACATTGTCGCA | | |
| | GTGACACTTCCAAAGCAGC | | |
| | TATTGTATTCTGGAGCCTC | | |
| | CTGGATGAACATCAGCTTT | | |
| | ATCACCTGGAGCCTGTTAC | | |
| | AAACGTAGAATCTTGACCT | | |
| | CAGCCCAGCCCTACTGAAT | | |
| | CAGAGTCTACATTTTTAAT | | |
| | GAGATCCCCANGTGATTCG | | |
| | CAAGCACATGAGAGTTTGA | | |
| | GAAGTGCTGTCTGGTTACT | | |
| | TATGGCAGCCTAATGACGG | | |
| | TAATCGAATT | | |
| 64 | Homology to TADA1 | | RF |
| | protein mRNA | | |
| | TGCTGCACNAGTCAATCAC | | |
| | ANTTTGCAACTGCAAAAGA | | |
| | GGCATAAACTGACGGGGAT | | |
| | CTCCTGCTTTACTATGCAC | | |
| | ACACAGCTTAACTCTCCAA | | |
| | CCGTGGAGAACAACTAACT | | |
| | CTATANGAAAAATNAAAAG | | |
| | NTAAACTNAACCATCCAAG | | |
| | GATGGGGGNAAGGGCTTCA | | |
| | TGGGTAACTTCTGGCCTGT | | |
| | NAGCACTAAATTNCAAACT | | |
| | GCTCCTGCAAGAAACCTNA | | |
| | AGGGCTGGGAAGGCTTTAT | | |
| | CTGGCTCAAAAACTAAGGN | | |
| | TGCTCTCNAAAGAATTCAA | | |
| | ATTCTCCTCAACAANCNAN | | |
| | AAANANTKGTCAATCCAAT | | |
| | TCAAGGCTCCCNTNANATT | | |
| | GNAAGNANTTCCTNTNAAC | | |
| | AACAACAAGGTGACTKACT | | |
| | YCTKCAACAATYTAAATTC | | |
| | CCCCGGCSNCCATGGGGGG | | |
| | CNGGAAANATSSAANTNTN | | |
| | GGCCCAATTTCCCCANAAT | | |
| | KAGTCTTATAAAAATCACW | | |
| | GGGCGTCTTTAAANGTCTK | | |
| | ACTGGGNAAAAAC | | |
| 65 | Homology to REX | AE003829 | RF |
| | mRNA for perixisome | | |
| | biogenesis factor | | |
| | (peroxin) | | |
| | CCATTCGTCCGTAGGTCCA | | |
| | GGTGTTTTACGTCAGCAGG | | |
| | GAAATGTGGCACACGCCCT | | |
| | CGAGGCATTTTAACACTGC | | |
| | GCTTCAGGAAATCTCAAGT | | |
| | TCCATCTTGTGTTAGTAAC | | |
| | GTACCCACATTTTGCTGGA | | |
| | GTTAGTTTATTAAAGATGC | | |
| | CTACGGTGAACTCTCTGGC | | |
| | GCAGGTTAAATGCAGTTTT | | |
| | GAAAACCTGGAAACATCAA | | |
| | ATGGAGGCGGGAAATAAGC | | |
| | TGGGGCCGAGCTGAGGGGC | | |
| | TGAACACGA | | |
| 66 | Homology to human | AL049761 | RF |
| | DNA sequence from | | |
| | clone RP5-863C7 on | | |
| | chromosome 20p12 | | |
| | TTGTGCTTAAAAAAATAGG | | |
| | CTTCCTCAGTGAAGCACCT | | |
| | GATAAAMTTAGGTGGTTGG | | |
| | ATTACAGTGGAAGCTCCAC | | |
| | TTTACACACACACACAAAC | | |
| | ACACAAAAACTGGCGACTT | | |
| | TTTCCATCCCCAATCCCTG | | |
| | CAMTGSTGAGACACAAAAT | | |
| | GAGTTTTWTGGCAAAGGAT | | |
| | CCTTAATCCCCAGAGACGC | | |
| | TTTGGCTTGTGGTGCTTTT | | |
| | TTTTTTAAGGCCTCTCTGC | | |
| | TCTGCCCGGTACCATGGGT | | |
| | CGAACGAGGGGTGTATAAA | | |
| | ATGGGGGCCTTGGGAAGCC | | |
| | TCCACGGTACAGGGCTGCA | | |
| | GGCCCCTCAGATGTGAACA | | |
| | CGACGACCTGGGA | | |
| 67 | Human cDNA | AK000407 | RF |
| | FLJ20400, clone | | |
| | KAT00587 | | |
| | TGTCAGGGATACCGAGAGC | | |
| | CGGTGTTCTGTTAACCTCT | | |
| | GGGAATGGTCCTCCCAATC | | |
| | CTAGGCCAGTTGGCCTGGG | | |
| | CCCAGGACCAAACCCCAAT | | |
| | CTGAGATCAGGCTTTTTAG | | |
| | GGACAAACCCTGCCCCCAG | | |
| | GTCAGGTGTGTTTCCAGGC | | |
| | CCAGGCCTTGGGCCCAACC | | |
| | CAAGACCAAGTGGCCTGGG | | |
| | CCCAGGCCCTAATCTANAT | | |
| | RCCAGAGCAGGTGGCCTCT | | |
| | TGGGCACAGGATCTGGTCT | | |
| | TAACTTAAGAATGGCTGGA | | |
| | CCTCAAGGCCTCGATCTTG | | |
| | CCCCCATTCTAAGAGCAGC | | |
| | AGGTCTTTTAGGAGCAAAT | | |
| | TCAGCTTCTTTCTCACAGG | | |
| | CTTCTGGAAACATGGGCAC | | |
| | AAGCCCATCCTCCATGGCA | | |
| | AGAGTACCTGGCCCCATGG | | |
| | GCCCAAACTCGGGTCCTAN | | |
| | CTCTCGGTATCCCTGACAA | | |
| | TCGAA | | |
| 68 | EST55810 from in- | AA349232 | RF |
| | fant adrenal gland- | | |
| | 2 | | |
| | TCGGATCCACAGAGACCTG | | |
| | CATGGGAGGTGGGGCCACA | | |
| | GGTCTGGTATCAGGCAAAC | | |
| | CTAGGTTGGAACACTGGCT | | |
| | CCATAAAGAGGAAGTCACT | | |
| | TAACCTTCTCTGGGGCATG | | |
| | GTTTCTTCATCTGTTCCCA | | |
| | CCTCTGAAGACTATCGTAA | | |
| | GACAGAATGAAAGTTAAGC | | |
| | AACTTAACGCAACGCCCAG | | |
| | GATACCAGAATTATTCTAA | | |
| | ATGGCAGAATCCTACTTAG | | |
| | TCTGTCATCTTGGGAGTTC | | |
| | TCTAGGCAGGCAGGTTGCC | | |
| | AGGGGTGGGGCTGAGATCC | | |
| | AGATGTGCTCTCGGTATCC | | |
| | CTGACA | | |
| 69 | Hu. mitochondrial | J01415 | RF |
| | DNA, frag. M1 | | |
| | J01415 | | |
| | TGACACTAGTCACGTGGCA | | |
| | GGGGGTTTTATATTGATAA | | |
| | TTGTTGTGATGAAATTGAT | | |
| | GGCCCCTAAGATANAGGAN | | |
| | ACACCTGCTAGGTGTAAGG | | |
| | AGAANATGGTTAGGTCTAC | | |
| | GGAGGCTCCAGGGTGGGAG | | |
| | TANTTCCCTGCTAAGGGAG | | |
| | GGTAAACTGTTCAACCTGT | | |
| | TCCTGCTCCGGCCTCMACT | | |
| | ATACCAAWTGCNACCAGGA | | |
| | TTAGGAAAAAGGGGGGTAA | | |
| | NATCAAAACCTTATNTTNT | | |
| | TTATGCGGGGAAACNCCAT | | |
| | ATCGGGGGCACCKATTATT | | |
| | AGGGGAACTATTCATTTCC | | |
| | AAANCCNCCGATTATGATG | | |
| | GGTATTACTATGAANAANA | | |
| | TTTTACAAATGCATGGGCT | | |
| | GTGACAANAACNTTRTAAA | | |
| | TTTGSNCTTACCNAAAANG | | |
| | TTNCCTGGGYTGGCCCANC | | |
| | TCGGCTCNAAAAANGAGKC | | |
| | TAAAACTTTTCCTAAGACN | | |
| | CCACCTCATGCCCCNAAAA | | |
| | AAAAGTANAGTNTTCCAAT | | |
| | TTTTTTGTGGNTTGKAAAA | | |
| | AAAAATCAACGGTCGGSAA | | |
| | AAWY | | |
| 70 | Hu. satellite rep. | X14302 | RF |
| | DNA; alphoid | | |
| | (X14302) | | |
| | TCGACGCTNAGAGTGGACA | | |
| | TTTGGAGCGCTTTCAGGAC | | |
| | GACGGTGAAAATGGAAATA | | |
| | TCTTCCAAGAAAATCTAGA | | |
| | TAGAAGCAATGTCAGAAAC | | |
| | TTTTATGTGATGGATCTAC | | |
| | TCAGCTAACAGAGTTGAAC | | |
| | CTTTCTTTTGAGAGAGCAG | | |
| | TTTTGCAACACTCTTTTTG | | |
| | TGGAATATGCAAGTGGATA | | |
| | TTANGGCAGCTTTGAGGAT | | |
| | TTCGTTGGAAACGGGAATA | | |
| | CATGTAAAAAGCAGACAGC | | |
| | AGCATTCTCAGAAACTTCT | | |
| | TTGTGATGTTTGCATTGAA | | |
| | GTCACANAGTTNAACATTC | | |
| | CCTTTGAGAGAGCAAGTTT | | |
| | GAAACACGCCTTTTGTCAT | | |
| | ATCTGGAAGTGTCCACTCT | | |
| | CANCGTCGAATCGAANTCC | | |
| | CGCNGGCGCCATGGNGGGC | | |
| | GGGAGCATGCNACTCNGGC | | |
| | CCAAATCGCCCTANANTGA | | |
| | GTCGTAATANAAATCACTG | | |
| | GCCGTCGTTTTACAACGTC | | |
| | TGACTGGGGAAAAACCCCC | | |
| 71 | Homo sapiens cDNA | AK001732 | RF |
| | FLJ1087O fis, clone | | |
| | NT2RP4001679 | | |
| | ATGNATACGACTCCTATAG | | |
| | GGCGAATTGGGCCCGACGT | | |
| | CGCATGCTCCCGGCCGCCA | | |
| | TGGCGGCCGCGGGAATTCG | | |
| | ATTACCGTCACTAGGCTGC | | |
| | CAAGTAGCTGGGACTACAG | | |
| | GCGCATGCCAGCATGCCTG | | |
| | GCTAATTTTTGTATTTTTA | | |
| | GTAGAGCGGGGGTTTTGCC | | |
| | ATGTTGGCCAGGCTGGTCT | | |
| | TGAACTCCTGACCTCAGGT | | |
| | GATCCGCCTGCCCTGGCCT | | |
| | CCCAAAGTGCTGGGATTAC | | |
| | AGGCATGAGCCACTGCGCC | | |
| | CAGCCTGTAATTTACCTTT | | |
| | GATGGTGACCTGAAATGCA | | |
| | GTATGATCCTATATGTGGC | | |
| | ACGTAGTAATGTTCAGTGT | | |
| | TTGCTGAATTAATTCTGGG | | |
| | CAGCCTAGTGACGGTAATC | | |
| | ACTAGTGAATTCGCGGGCC | | |
| | GCCTGCAGGTCGACCATAT | | |
| | GGGAGAGCTCCCAACGCCG | | |
| | TTGGATGCATAGCTTGAGT | | |
| | ATTCTATAGTGTCACCTAA | | |
| | ATAGCTTGGGCCGTAATCA | | |
| | TGGTCATAGCTT | | |
| 72 | QV4-BT0407 homo | BE070362 | RF |
| | sapiens DNA | | |
| | AGTTAGCCGCAGCGAAGAG | | |
| | GACCCAGCAGGAATCATGA | | |
| | GGGAAGGAAAATGCAGCAC | | |
| | TCTAAATGGCCACTCAGGC | | |
| | GTTCCTATTCACTCGGAAA | | |
| | ATTAGGTTCATTTCACAGG | | |
| | ACACAGCAGTGTAGATCAG | | |
| | GCTTCAACTTAACATTTAA | | |
| | GGGAAATGTCAGATTTTTT | | |
| | TTTAATTTAATGAAATTGT | | |
| | TAATGAGGAAAAATTTTTA | | |
| | ATATAGTCTTATCTACCAC | | |
| | ACATCCCCATAGATTTAAG | | |
| | GATTTTAATAGAAAGTCAT | | |
| | GATGTATGTATTCAAGCCA | | |
| | CGTTAAAAGAAAAAATATA | | |
| | ACTATGGACCGGTATTCAG | | |
| | TGAATACAGTTTCATGGTT | | |
| | TTTAATTCTTTCAAAGGCA | | |
| | CATTAAAAATGGTGTGCTG | | |
| | ATAAACCCCAAGTAAATTA | | |
| | ACCCTTTTTCCGTATAAAT | | |
| | CCATTTTTTGTTTTGAAGA | | |
| | GGGGAAATTATATTTATTG | | |
| | GTTGTTTACTGGAATCCTG | | |
| | GTGTGGAAAGCATATCAGA | | |
| | TATGTATGAACTGCTACTG | | |
| | GCTGTACTTTCCGATTTAC | | |
| | GGGACATCAATTTTATTGG | | |
| | CTTATTTGTNANGANCGTG | | |
| | ATTAACCATGGACCATTGA | | |
| | AGTACCTATTTTAATGGTN | | |
| | GGGCCCTTCAANTGGGGAT | | |
| | TGGGNCCAGTGGCCCTTCA | | |
| | AGGTCTTCTTGGGGGATTC | | |
| | CGAATNCACTTNAGTGGAA | | |
| | TTTCNCNGGNCCGCCTTGC | | |
| | CAAGGTCGANCCATNTTNG | | |
| | GGAAAAGCTTCCAAACCGC | | |
| | CGTTTGGATTC | | |
| 73 | Homology to Soares | HS151B14 | RF |
| | ovary tumor NbHOT | | |
| | Homo sapiens cDNA | | |
| | clone IMAGE:756918, | | |
| | 5′. | | |
| | CCTTTNTAAACCGACGGCC | | |
| | ATTGAATTGTAATACGACT | | |
| | CACTATAGGGCGAATTGGG | | |
| | CCCGACNTCTCATGCTCCC | | |
| | GGCCGCCATGGCGGCCGCG | | |
| | GGAATTCNATTGTTACACC | | |
| | AACCGCCAGGGGGCCTTGG | | |
| | CCAGGTCAAGGGTTCTGTG | | |
| | AGGAAAGGACCCANGATTG | | |
| | TGGGGGCATTTGGGGGGTG | | |
| | AATTGGCCCCCNAAAAATG | | |
| | GAACCCACACCCATAGCTC | | |
| | TCCCCANANCTNATACGGC | | |
| | ATCCTGCNAAAANACCTGC | | |
| | CCTCCTCACTGGGATCCCC | | |
| | TTCCTGCCTCCTCCCAGGG | | |
| | CTCTGCCANGGCCTTGCTC | | |
| | AATCCCTTCCACCAAAGTC | | |
| | ATCTNAACTTCCNTTTCCC | | |
| | CAGGGCCTCCANCTGCCCT | | |
| | CAAACACTGATTTNTGTCC | | |
| | CCAGGTGCTCTCTGCCCCT | | |
| | CANGCCCCTCTCANCGGCC | | |
| | CANTGCCCCGACTCTCCAG | | |
| | GCTTTATCAAGGTGCTAAG | | |
| | GCCCGGGTGGGCANCTCCT | | |
| | CGTCTCAAAGCCCTCCTCC | | |
| | GGCCTGGTGCTGCCTTTAC | | |
| | AAACACCTGCAAGAAAANG | | |
| | GCACGGAAACCCCAAGCTT | | |
| | TAAAGCCCTCAACAAGTCT | | |
| | GGTGGATCCGAANCACTAA | | |
| | TGAATTCNCGGCCCCCTGC | | |
| | ANGTCNACAATATNGGAAA | | |
| | NCTCCCAACCCGTTTGATG | | |
| | CAAACTTGANTTTCTATAN | | |
| | TGTCACCTAAATNCTTGGG | | |
| | GTAATCATGGCAAAACTTT | | |
| | TCCCTNTTGGGNNATAA | | |
| 74 | Homology to | AA308048 | RF |
| | EST17889 Aorta en- | | |
| | dothelial cells, | | |
| | TNF alpha-treated | | |
| | Homo sapiens, | | |
| | zv23b05.r1 Soares_ | | |
| | NhHMPu_S1 Homo sa- | | |
| | piens cDNA clone | | |
| | IMAGE:754449 | | |
| | ACTAGGCKGACCACACCAA | | |
| | CTAGGATGWTCACCACAMC | | |
| | CMCCAGGATGACAATGAAS | | |
| | CTGATAACACCAAATGCAS | | |
| | CCMCAGTKAGCACANTYTC | | |
| | TGACNTGGGGCTGGGCARG | | |
| | ATGGTCGCATCTTCCCTCA | | |
| | WGCTCARGSTCAWGGTGGT | | |
| | TGAATTGGGGGGAACARTT | | |
| | TGAAATGTGTCTGGGGTAC | | |
| | CAATGCTGGACAAATGGCT | | |
| | TGGCCTGTTAACCTCTGAG | | |
| | GAAGGGATNTTCCTGGGTT | | |
| | GGAANAAACTGGCT | | |
| 75 | Homology to EAP30 | AF156102 | RF |
| | subunit of ELL com- | | |
| | plex (EAP30), mRNA | | |
| | GCTGCACGAGTCAGTCACA | | |
| | CTTCGATAATTTGGACACC | | |
| | TAGTTCGTAATAGAAGTCC | | |
| | CCCACGCCCAGCATCTCAG | | |
| | ACCAAAATCCTTTTCCAGC | | |
| | TACAAGACAGAAAAACAAA | | |
| | ATCTCGGTTATTGATTAAT | | |
| | TTACATCCCTTCTTTTCTT | | |
| | TCAAACCTGCATTAAAACA | | |
| | TCTATCACATGGGAAGTCT | | |
| | TCCCTGCATGGCATGAAGT | | |
| | TCTTCTGCTCCATCTGCTA | | |
| | CTCAGGAAACAATCGGACT | | |
| | CACGGTAACTCCTGCTGAA | | |
| | GAACAGGGTCAGCCTAGTG | | |
| | ACGG | | |

The genes identified were relevant genes for vascular endothelial cells and in some cases consistent with the known effects of estrogen in the cardiovascular system. One such gene, prostacyclin I2 synthase, was upregulated 4 fold by estradiol at 24 hours (FIG. 2). This enzyme is required for that last step in the synthesis of prostacyclin (PGI2). PGI2 has been shown to induce vasodilation, inhibit smooth muscle proliferation and platelet aggregation. This indicates that PGI2 has atherogenesis inhibiting properties. Another relevant gene was natriuretic peptide receptor B (NPR-B) which is the receptor for C-type natriuretic peptide (CNP), an endothelium dependent vasodilator.

Further experiments demonstrated that these two genes were not regulated by estrogen in MCF-7 cells, an ER positive breast cancer cell line. Several other genes were examined for estrogen regulation in MCF-7 cells and were found to be regulated by estrogen in MCF-7 cells (ATP sulfurylase/APS kinase, podocalyxin), but others were either not expressed (connexin 37, diastrophic dysplasia sulfate transporter) or not regulated by estrogen in a similar fashion in MCF-7 cells (clusterin). This demonstrates the usefulness of the VE-ERα cells in identifying genes regulated by estrogen in a cell type specific manner.

### Example 3

Effect of Estrogen and Certain Estrogen-Related Compounds on Cyclin A1 Expression

This Example illustrates the effect of 17-β-estradiol (E₂) and diethylstilbestrol (DES), an estrogen agonist, on cyclin A1 expression.

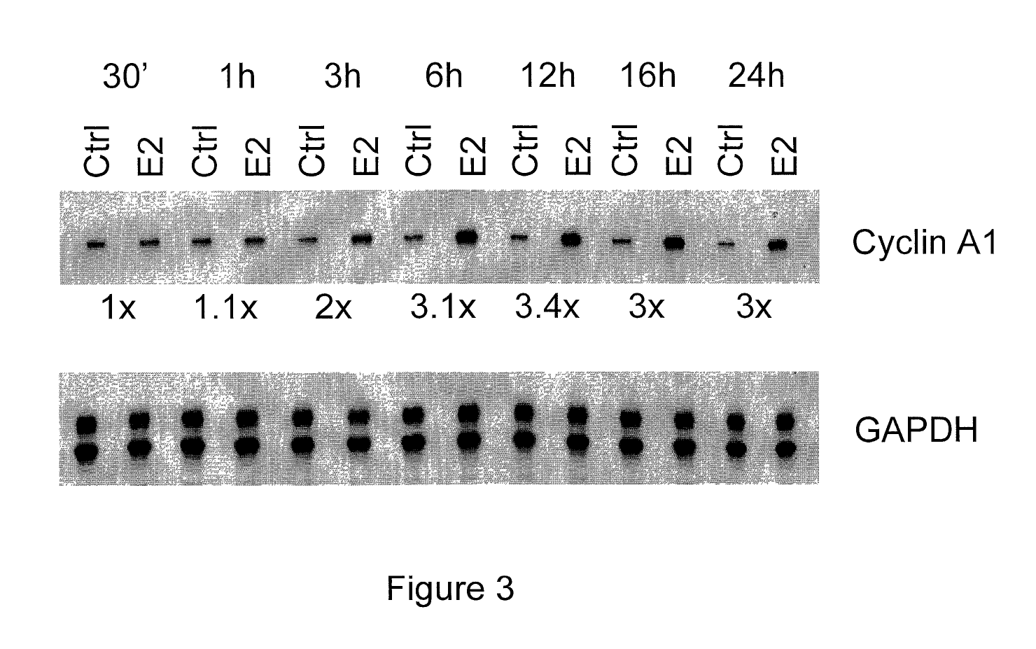

ERa enhanced cells as described above were treated by adding 0.1% ethanol (ctrl) or 100 nM 17-β-estradiol (E2) for various times. RNA was prepared using standard methods and RNase protection experiments were performed to evaluate the time course of cyclin A1 induction. FIG. 3 shows the mRNA levels of cyclin A1 at the indicated times, with glyceraldehyde-3-phosphate dehydrogenase (GADPH) levels also provided for reference. The results demonstrate that cyclin A1 mRNA is increased by treatment with estradiol within 3 hours.

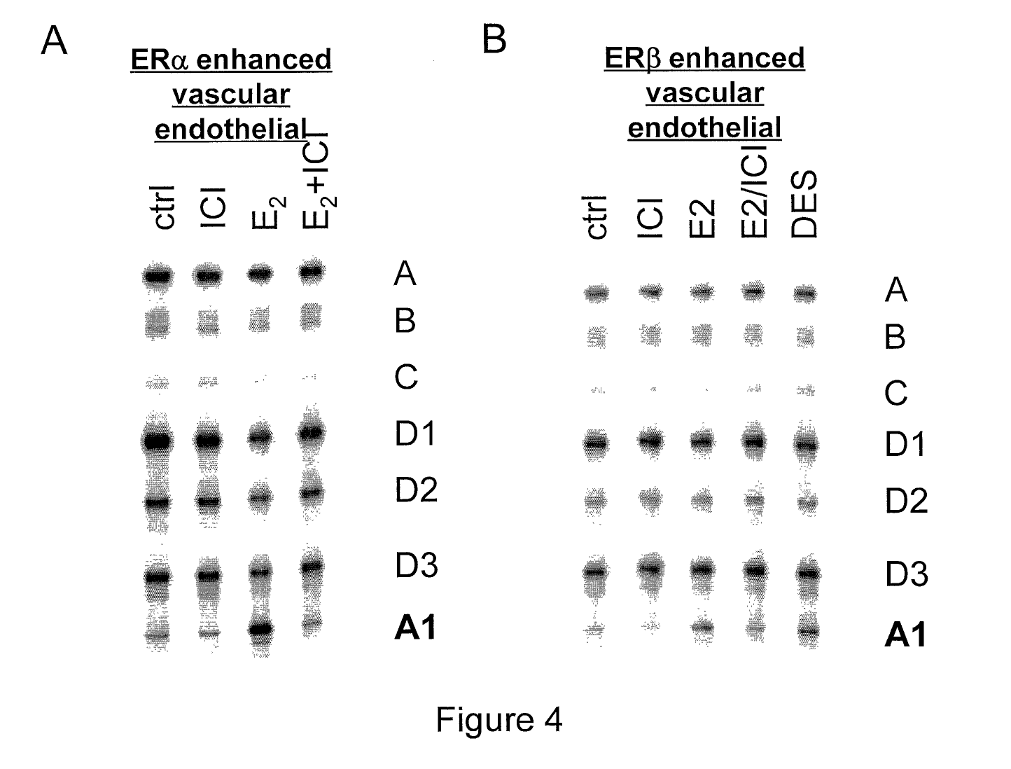

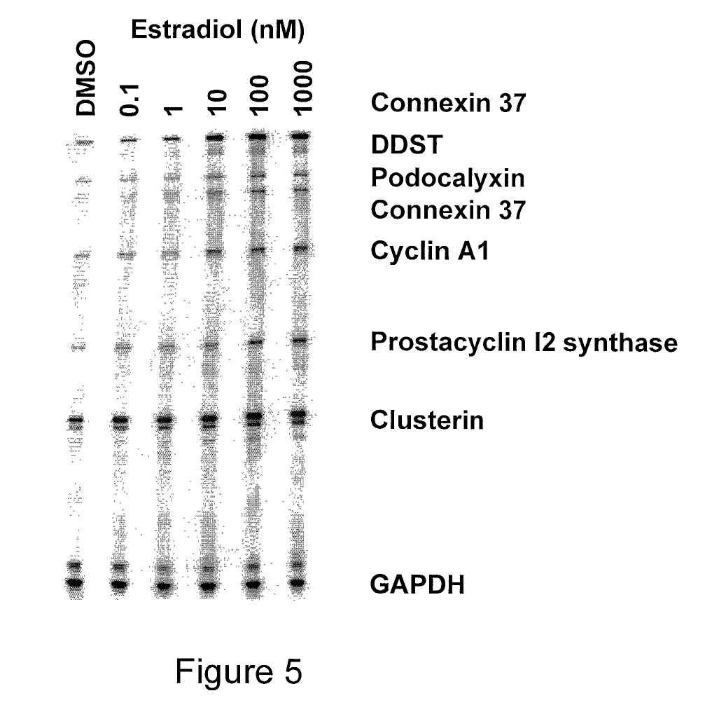

In a similar experiment, RNase protection assays were performed using estrogen receptor α- and estrogen receptor β-enhanced vascular endothelial cells. The estrogen receptor β-enhanced cells were prepared as described above for estrogen receptor a-enhanced cells, except that the retroviral vector expressed estrogen receptor β (Kuiper, G. PNAS 1996 93:5925-5930) Cells were treated by adding 0.1% ethanol (ctrl), 100 nM 17-β-estradiol (E2), 1 μM ICI 164384 (ICI), or 10 nM diethylstilbestrol (DES), and RNase protection assays were performed as described above. The results are presented in FIGS. 4A and 5B, which show the levels of various cyclins in estrogen receptor α- and estrogen receptor β-enhanced cells, respectively. Arrows indicate mRNA for each indicated cyclin. These results demonstrate that the estradiol induction of cyclin A1 mRNA can occur with both estrogen receptor α- and estrogen receptor β-enhanced vascular endothelial cells. The “pure” estrogen antagonist ICI 164384 inhibits this response, whereas the synthetic estrogen DES mimics the estradiol effect. Cyclin A1 is the only cyclin in the panel increased by estradiol treatment.

Cyclin A1 expression and the effect of estradiol on expression in a number of immortalized and primary cells lines was further evaluated as described above. These results are shown in Table II, in which (+) indicates detectable cyclin A1 whereas (−) indicates no detectable cyclin A1 expression. These results demonstrate that although cyclin A1 is detectable in some other cell types, cyclin A1 expression is only increased by estradiol in the vascular endothelial cells with enhanced estrogen receptor α- and estrogen receptor

TABLE II
Cyclin A1 Expression and Induction in Various Cell Lines

| Cell Line | Control | Estradiol |
|---|---|---|
| ERα-enhanced vascular | + | +++ |
| endothelial* | | |
| ERβ-enhanced vascular | + | ++ |
| endothelial* | | |
| Vascular endothelial* | + | + |
| Primary vascular endothelial | + | + |
| Mammary epithelial* | + | +/− |
| MCF-7 (mammary cancer cell | − | − |
| line) | | |
| Endometrial epithelial* | + | + |
| Osteoblasts* | − | − |
| Vascular smooth muscle* | − | − |
| Primary vascular smooth muscle | − | − |

*Immortalized human cells

### Example 4

Use of Estrogen-Regulated Markers for Identifying Agents with Estrogenic Activity

This Example illustrates screens for estrogenic compounds using estrogen-regulated markers.

An RNase protection assay (RPA) was used to examine a panel of genes identified as estrogen-regulated in VE-ERα cells. The reference compounds 17-β-estradiol, 17α-estradiol (inactive estrogen) ICI 164 384, tamoxifen, and raloxifene and were tested in a multigene assay with VE-ERα cells (5 dose—0.1 nM, 1 nM, 10 nM, 100 nM, 1000 nM for 24 hours). As expected, 17-β-estradiol induced expression of representative estrogen-regulated genes (indicated in FIG. 5). ICI 164,780, a compound generally regarded as a pure estrogen antagonist, showed no induction of gene expression and lowered basal expression of some genes at higher concentrations. Raloxifene and tamoxifen showed no clear induction of any of the genes. In fact, raloxifene had similar effects to ICI 164,780, repressing rather than inducing gene expression.

In a further experiment, compounds known to have estrogen-like activity based on ER binding and ERE transactivation (ERE luciferase reporter in Chinese hamster ovary cells expressing transfected ERα) were evaluated. The data with these compounds showed a strong correlation between ER binding activity and ERE transactivation. In general, the compounds that bound strongly to ER also were strong activators of transcription, judged by luciferase activity in the reporter assay. When these compounds were examined in the multigene RPA, some compounds showed results correlating with the ER binding and transactivation results (Table III, compounds D and E). However, a number of compounds which were highly active in ER binding and in the ERE transactivation assay had very low activity in the multigene assay (Compounds A, B, C). In addition, several compounds which had significant ER binding and minimal ERE-luciferase transactivation were strong activators in the multigene assay (Compounds F and G). This suggests that the assay can detect compounds that are activating through promoter elements other than EREs. In order to confirm that these active compounds were working in an ER dependent manner, VE103-ERα cells were treated with a single dose of compound and increasing doses of ICI 164,384. The results of this experiment demonstrated that ICI 164,384 was able to inhibit competitively the activation of these genes, indicating ER dependence. Table III shows the results of compounds tested on prostacyclin I2 synthase in the VE-ERα cells compared to ER binding and ERE transactivation assay. All results are presented as percent of estrogen. PGI2 synthase numbers were based on EC50 from a 5-point dose response like that shown for estrogen in FIG. 5.

TABLE III
Results of Compounds tested on Prostacyclin I2 Synthase in
the VE-ERα Cells Compared to ER Binding and ERE
Transactivation Assay

| | Detected In Chinese Hamster | | |
|---|---|---|---|
| | Ovary Cells | | VE-ERα Cells |
| | | ERE | PG I2 |
| Compound | ER Binding | transactivation | synthase |
| 17-β-estradiol | 100 | 100 | 100 |
| A | 199 | 63 | 6.5 |
| B | 170 | 55 | 2.3 |
| C | 74 | 130 | 8 |
| D | 197 | 70 | 129 |
| E | 103 | 101 | 97 |
| F | 6.2 | 0.4 | 224 |
| G | 2.2 | 0.2 | 90 |

From the foregoing, it will be appreciated that, although specific embodiments of the invention have been described herein for the purpose of illustration, various modifications may be made without deviating from the spirit and scope of the invention. Accordingly, the present invention is not limited except by the appended claims. Various publications are cited herein, the disclosures of which are incorporated by reference in their entireties. Various nucleic acids are referred to by accession number herein, the sequences of which are incorporated by reference in their entireties.

1. A method for evaluating the cardiovascular effect of a candidate agent, comprising the steps of:
(a) contacting an estrogen receptor-enhanced vascular endothelial cell with a candidate agent; and
(b) determining whether the candidate agent modulates expression of at least one estrogen-regulated marker, wherein the expression of the marker is previously determined to be induced by estrogen in estrogen receptor-enhanced vascular endothelial cells, and therefrom determining whether the candidate agent has a cardiovascular effect.

2. A method according to claim 1, wherein the cell is immortalized.

3. A method according to claim 1, wherein step (b) comprises the steps of:
(i) detecting a level of an estrogen-regulated marker; and
(ii) comparing the detected level of the estrogen-regulated marker with a level of the estrogen-regulated marker in the absence of the candidate agent.

4. A method according to claim 1, wherein step (b) comprises the steps of:
(i) detecting a level of mRNA encoding an estrogen-regulated marker; and
(ii) comparing the detected level of mRNA encoding the estrogen-regulated marker with a level of mRNA encoding the estrogen-regulated marker in the absence of estrogen-related compound.

5. A method according to claim 4, wherein the level of mRNA is detected using an Rnase protection assay.

6. A method according to claim 4, wherein the level of mRNA is detected by:
preparing cDNA from mRNA present within the cell; and
amplifying mRNA encoding the estrogen-regulated marker via polymerase chain reaction.

7. A method according to claim 6, wherein the amplification is performed using real-time PCR analysis.

8. A method according to claim 1, wherein the cell is transfected with a polynucleotide comprising a promoter for an estrogen-regulated marker, operably linked to a reporter gene.

9. A method according to claim 8, wherein step (b) comprises the steps of:
(i) detecting a level of reporter protein activity; and
(ii) comparing the level of reporter protein activity detected with a level of reporter protein in the absence of candidate agent.

10. A method according to claim 8, wherein step (b) comprises the steps of:
(i) detecting a level of reporter mRNA; and
(ii) comparing the level of reporter mRNA detected with a level of reporter mRNA in the absence of estrogen-related compound.

11. A method according to claim 1, wherein the ability of the candidate agent to modulate expression of at least two estrogen-regulated markers is determined.

12. A method according to claim 1, wherein the estrogen-regulated marker is selected from the group consisting of SEQ ID NOs:1-74.

13. A method for identifying a selective estrogen receptor modulator (SERM), comprising the steps of:
(a) contacting at least two different cells that express an estrogen-regulated marker expression with a candidate SERM;
(b) determining levels of expression of at least one estrogen-regulated marker in the cells; and
(c) comparing the levels of expression with levels of expression of the estrogen-regulated marker(s) in cells following contact with estrogen, and therefrom determining whether the candidate SERM is a selective estrogen receptor modulator.

14. A method according to claim 13, wherein the estrogen-regulated marker is selected from the group consisting of SEQ ID NOs:1-74.

15. A method according to claim 13, wherein levels of at least two estrogen-regulated markers are determined.

16. A method according to claim 13, wherein each of the cells is independently selected from the group consisting of bone, urogenital, cardiovascular, breast, ovarian, endometrial, and central nervous system cells.

17. A method according to claim 13, further comprising the steps of:
(d) contacting breast cells that express an estrogen-regulated marker expression with estrogen and a candidate SERM;
(e) determining levels of expression of at least one estrogen-regulated marker in the breast cells; and
(f) comparing the levels of expression with levels of expression of the estrogen-regulated marker(s) in the breast cells following contact with estrogen, in the absence of candidate SERM.

18. A method for inhibiting the development of a cardiovascular disease in a patient; comprising administering to a patient an agent identified according to the method of claim 1 or a SERM identified according to claim 13.

19. A method for inhibiting the development of breast cancer in a patient; comprising administering to a patient a SERM identified according to the method of claim 17, wherein the SERM inhibits induction of estrogen-regulated marker expression by estrogen in breast cells.

20. A method for inhibiting the development of osteoporosis in a patient; comprising administering to a patient a SERM identified according to claim 13.

21. An isolated or recombinant nucleic acid comprising SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; or SEQ ID NO: 75.

22. An isolated or recombinant nucleic acid that hybridizes under stringent conditions to the nucleic acid of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; or SEQ ID NO: 75.

23. An isolated or recombinant nucleic acid that is at east 75% identical to at least 50 consecutive nucleic acids of SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; or SEQ ID NO: 75.

24. The nucleic acid of claim 23, that is at least 85% identical to at least 50 consecutive nucleic acids SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; or SEQ ID NO: 75.

25. The nucleic acid of claim 24, that is at least 95% identical to at least 50 consecutive nucleic acids SEQ ID NO: 56; SEQ ID NO: 57; SEQ ID NO: 58; SEQ ID NO: 59; SEQ ID NO: 60; SEQ ID NO: 61; SEQ ID NO: 62; SEQ ID NO: 63; SEQ ID NO: 64; SEQ ID NO: 65; SEQ ID NO: 66; SEQ ID NO: 67; SEQ ID NO: 68; SEQ ID NO: 69; SEQ ID NO: 70; SEQ ID NO: 71; SEQ ID NO: 72; SEQ ID NO: 73; SEQ ID NO: 74; or SEQ ID NO: 75.
